(19) United States
(12) Patent Application Publication
(10) Pub. No.: US 2025/0381547 A1
(43) Pub. Date: Dec. 18, 2025

(54) APPARATUS, SYSTEM AND METHOD USING NONTHERMAL PLASMA

(71) Applicant: SpiralWave Inc., SAN FRANCISCO, CA (US)

(72) Inventor: Abedelazeem M. A. BUKHARI, WÖRGL (AT)

(21) Appl. No.: 19/237,458

(22) Filed: Jun. 13, 2025

Related U.S. Application Data
(60) Provisional application No. 63/660,060, filed on Jun. 14, 2024.

(51) Int. Cl. B01J 19/08; B01D 53/02; B01D 53/14; B01D 53/32; B01J 19/12

(52) U.S. Cl. CPC B01J 19/088; B01D 53/32; B01J 19/122; B01D 53/02; B01D 53/14; B01D 2257/302; B01D 2257/404; B01D 2257/502; B01D 2257/504; B01D 2258/06; B01D 2259/124; B01D 2259/818; B01J 2219/00045; B01J 2219/0805; B01J 2219/0869; B01J 2219/0884; B01J 2219/0896; B01J 2219/1203

(57) ABSTRACT

An apparatus comprises a chamber, an emitter configured to emit electromagnetic radiation into the chamber, and an igniter configured to provide energy at an ignition point within the chamber to initiate a gas discharge. The emitter and the igniter are operable in conjunction to generate nonthermal plasma within the chamber at atmospheric pressure.

### CROSS-REFERENCE TO RELATED APPLICATION

This application claims the benefit of U.S. Provisional Application Ser. No. 63/660,060 filed Jun. 14, 2024, which is hereby incorporated by reference herein in its entirety.

### TECHNICAL FIELD

The present invention relates to an apparatus, system and method using nonthermal plasma. Particular aspects of the present invention may relate to capturing substances, for example as carbon dioxide, from air using nonthermal plasma.

### TECHNICAL BACKGROUND

It remains uncertain whether conventional processes for capturing greenhouse gases, such as CO2, will succeed to meet the 1.5° C. global warming pathway set forth by the Intergovernmental Panel on Climate Change (IPCC). The conventional processes often rely on chemical processes, use consumables, employ pre- or post-treatments or pressurization means, require cooling or heating, have high space requirements, or consume natural materials. As such, conventional processes for capturing greenhouse gases are often accompanies by environmental drawbacks.

For instance, WO 2009/048242 A2 describes a method involving nonthermal plasma for CO2 decomposition. Bogaerts et al.: “Plasma Technology for CO2 Conversion” in Front. Energy Res., 7 Jul. 2020, Sec. Carbon Capture, Utilization and Storage, explores various methods for plasma CO2 conversion.

The inadequacy of conventional processes for capturing greenhouses gases such as CO2 calls for innovative solutions.

### SUMMARY OF THE INVENTION

The present invention is defined according to the subject matter of the appended independent claim(s). Particular embodiments are given by the additional features of the appended dependent claims.

Disclosed herein is an apparatus. The apparatus may comprise a chamber; an emitter and an igniter. The emitter may be configured to emit electromagnetic radiation into the chamber. The igniter may be configured to provide energy at an ignition point within the chamber to initiate a gas discharge. The emitter and the igniter may be operable in conjunction to generate nonthermal plasma within the chamber at atmospheric pressure.

The term apparatus as disclosed herein may generally refer to a device, a set of components or a set of devices designed and/or arranged to achieve a technical purpose or function. The apparatus may be implemented as a single device, in which case the term “apparatus” may be used interchangeably with the term “device”, or as an assembly of two or more devices. Specifically, the apparatus as disclosed herein may be configured for controlled generation and handling of nonthermal plasma at atmospheric pressure. Furthermore, the term “apparatus” may be used interchangeably with the term “system” as disclosed herein, unless otherwise indicated or technically inappropriate.

Within this disclosure, the term “apparatus” may encompass any integral and supporting element described herein, including, but not limited to, a chamber, an emitter, an igniter, a fluid injector, a filter, and associated controls. The apparatus is typically physically contained or integrated into a cohesive, operational unit designed for (nonthermal) plasma generation, interaction with gases (particularly air), and optionally, chemical reactions facilitated by plasma conditions.

The apparatus may include any one, some or all of the features of any of the apparatuses, systems or devices as described below, unless indicated otherwise or technically inappropriate. Moreover, any one, some or all of the features of any of the methods as disclosed herein may be applicable to the apparatus, unless indicated otherwise or technically inappropriate.

The emitter may be configured to emit electromagnetic radiation into the chamber. The emitter as disclosed herein may broadly refer to a device, a component, or a unit configured to generate electromagnetic radiation and/or direct electromagnetic radiation to a desired location or in a desired direction. Specifically, the emitter may be configured to produce electromagnetic radiation suitable for initiating and/or maintaining a nonthermal plasma at atmospheric pressure.

The emitter as disclosed herein may encompass one or more electromagnetic radiation sources selected from, but not limited to, a microwave source, such as a magnetron, a radio-frequency generator, an ultraviolet lamp, an infrared emitter, a laser, and an antenna. The emitter may be configured to emit electromagnetic radiation in a continuous manner, in a pulsed manner, or in a combination of both. The emitter may be configured to emit electromagnetic radiation within a spectral range corresponding to ultraviolet, visible, infrared, microwave, and radiofrequency. The emitter may be configured to emit electromagnetic radiation in a wavelength range from 100 nanometers to 1 meter.

In specific examples, the emitter may be configured to emit electromagnetic radiation at resonant frequencies of a target molecule, for example CO2, H2O or N2. In specific examples, the emitter may be configured to emit electromagnetic radiation in a pulsed manner and synchronized with an ignition event at an ignition point of the igniter as described herein. In specific examples, the emitter may be or comprise a continuous-wave (CW) microwave source and/or a pulsed solid-state microwave source, a laser, or a radio-frequency antenna. The emitter may be configured for optimized (nonthermal) plasma generation. In further examples, the emitter may be or comprise a magnetron configured to generate a microwave radiation, and a waveguide coupled to the magnetron, The emitter may be configured specifically for the chamber (adapted to) so as to generate standing electromagnetic waves in the chamber.

The igniter may include any one, some or all of the features of the igniter, charge carrier source or electron source as described below, unless indicated otherwise or technically inappropriate. The term “igniter” may be used herein in an interchangeable manner with the terms charge carrier source or electron source, unless indicated otherwise or technically inappropriate. Moreover, any one, some or all of the features of any of the methods as disclosed herein may be applicable to the emitter, unless indicated otherwise or technically inappropriate.

The igniter may be configured to provide energy at an ignition point within the chamber to initiate a gas discharge. The emitter as disclosed herein may broadly to a device, a component, or a system configured to deliver energy to the ignition point to initiate a gas discharge, resulting in plasma formation (plasma ignition). The igniter may be used to facilitate the generation of nonthermal plasma at atmospheric pressure. Hereinafter, the term “igniter” may be used interchangeably with the term “charge carrier source” or “electron source” unless indicated otherwise or technically inappropriate.

The igniter as used herein may encompass one or more mechanisms capable of initiating a gas discharge through a controlled input of energy at a precise location referred to as the ignition point. Such mechanisms may employ, without being limited to, an electron source (such as a high-voltage electrode pair, a spark gap, a Tesla coil, etc.), an electrical arc, a photo-ionization source (such as a laser, an ultraviolet lamp), or an electromagnetic field enhancement device. Specifically, the emitter may be or comprise a pair of high-voltage (i.e., at a voltage of 1000 Volts or higher) electrodes configured to generate a gas discharge therebetween. Additionally or alternatively, the emitter may be or comprise a tesla coil configured to generate high-frequency (e.g., 100 kHz to 300 GHZ) electrical fields capable of initiating avalanche ionization in gas molecules. Additionally or alternatively, the emitter may be or comprise a laser (device) configured to deliver focused electromagnetic radiation at the ignition point, inducing photoionization for plasma formation (plasma ignition). Additionally or alternatively, the emitter may be or comprise a spark gap device or a Marx generator configured to provide rapid electrical discharge pulses to ignite nonthermal plasma at atmospheric pressure.

The emitter may include any one, some or all of the features of the emitter, microwave emitting unit or high frequency emitting unit as described below, unless indicated otherwise or technically inappropriate. The term “emitter” may be used herein in an interchangeable manner with the terms microwave emitting unit or high frequency emitting unit, unless indicated otherwise or technically inappropriate. Moreover, any one, some or all of the features of any of the methods as disclosed herein may be applicable to the emitter, unless indicated otherwise or technically inappropriate.

The term “ignition point,” as used herein, may refer to a defined spatial location or region where energy is provided, delivered or concentrated by the igniter, thereby initiating a gas discharge. In particular, the igniter and the chamber may be disposed in a manner that the ignition point lies within the chamber. The ignition point may refer to a designated spatial location where the initial ionization by the igniter occurs. As discussed herein, the ignition point may be positioned in alignment with an electromagnetic field maximum generated by the emitter.

The emitter and the igniter may be operable in conjunction to generate (and sustain) nonthermal plasma, i.e., plasma conditions without reaching or approaching thermal equilibrium. Accordingly, the emitter and the igniter may be used in combination to create and sustain a nonthermal plasma within the chamber at atmospheric pressure. The combined operation of the emitter and the igniter may encompass at least one of: mutual compatibility, synchronization of operation, and optimization of both energy inputs. The combined operation of the emitter and the igniter may allow for generation of stable and reproducible (nonthermal) plasma and maintenance without necessitating pressure decrease or supply of a noble gas.

For example, the igniter may provide localized energy input (e.g., free electrons, sparks, arcs, or focused radiation), thereby initiating the gas discharge by creating the first electrons or charged particles essential for plasma ignition. Simultaneously or subsequently, the emitter (e.g., microwave source) may provide electromagnetic radiation tuned to excite gas molecules (such as CO2, N2, or any other air constituent(s)) resonantly, raising their energy states to facilitate ionization. Thus, the emitter and the igniter may be operable in conjunction (e.g., in a synchronized manner) so as to create and sustain a nonthermal plasma.

In a specific example, the emitter may be or comprise a microwave emitter (e.g., a magnetron at 2.45 GHZ) configured to generate standing electromagnetic waves within the chamber, precisely timed with the igniter that may be or comprise an electron source (e.g., a pair of high-voltage electrodes creating an electron-rich spark) configured to provide free electrons at the ignition point that, for example, coincides with the wave's antinode. Such a configuration may be capable of initiating and sustaining nonthermal plasma.

In a further specific example, which may be combinable with any other example(s) disclosed herein, the emitter may be or comprise a solid-state microwave emitter configured to produce pulses precisely coincident with the igniter that may be or comprise a spark gap device or a Marx generator, thereby providing simultaneous resonance excitation of gas molecules, such as CO2, and immediate avalanche ionization and forming nonthermal plasma under atmospheric conditions.

In a further specific example, which may be combinable with any other example(s) disclosed herein, the igniter may be or comprise a laser device configured to deliver pulses of light at the exact spatial position and timing of maximal electromagnetic radiation intensity provided by the emitter, which may be or comprise a microwave device, a radio-frequency antenna, or any other suitable device. Such a combined operation of the emitter and igniter may achieve inducing ionization without substantial heating, thereby generating and sustaining nonthermal plasma.

The chamber may enable controlled formation and maintenance of non-thermal plasma by confining electromagnetic energy and ionized gases. The chamber may be configured to permit airflow, e.g., through at least one of: one or more inlets, one or more outlets, or a gas-permeable sidewall, so that ambient air or other gases can pass through or remain confined for reaction with the plasma. The chamber geometry (e.g., cylindrical, columnar, beam-shaped, polygonal, etc.) may be adapted to support standing electromagnetic waves and/or enhancement of electromagnetic field(s). Such specific chamber geometry may contribute to increasing energy transfer efficiency, especially when the emitter is tuned to generate resonant field conditions. The chamber may have a conductive sidewall (or conductive sidewalls) that may serve as a Faraday shield, while partially open or perforated sections may allow gas exchange or optical access. Herein, the expressions “airflow” and “gas flow” may be used interchangeably unless indicated otherwise or technically inappropriate.

In particular examples, the chamber may be implemented as a metal tube with conductive sidewalls and arranged to extend in a direction parallel or substantially parallel to the gravitational vector (i.e., arranged vertically). In this configuration, air or other gases may ascend within the chamber due to a slight temperature increase caused by interaction with the plasma. Although the plasma remains essentially at ambient temperature, as is characteristic of nonthermal plasma, localized heating may nonetheless occur. This temperature gradient can induce buoyant upward flow of the gas. At the same time, cooler ambient air or other gases may enter the chamber through gas-permeable sidewalls, or via one or more designated openings or gaps in the chamber wall. Depending on the geometry and arrangement of these openings or gaps, the resulting airflow within the chamber may follow a substantially spiral or helical path in the upward direction.

The term “chamber”, as used herein, may refer to one or more structural components and a volume at least partially surrounded by the one or more structural components. The chamber may be configured so that nonthermal plasma can be generated and sustained at atmospheric pressure within the chamber. The chamber may be configured to be gas-permeable.

The chamber may include any one, some or all of the features of the chamber, air chamber system or plasma chamber as described below, unless indicated otherwise or technically inappropriate. The term “chamber” may be used herein interchangeably with the terms air chamber system or plasma chamber, unless indicated otherwise or technically inappropriate. Moreover, any one, some or all of the features of any of the methods as disclosed herein may be applicable to the emitter, unless indicated otherwise or technically inappropriate.

According to the subject matter disclosed herein, the emitter is configured to provide ng electromagnetic radiation tuned to resonate with gas molecules (e.g., CO2, N2 or any other air constituent(s)), thereby effectively preparing gas molecules to become ionized by raising their vibrational energy levels. The igniter then provides focused, localized ionization (electron injection, spark, or photon-induced ionization) at the most energy-efficient moment or spatial location, dramatically reducing the energy required for initiating plasma compared to independent operation. The electromagnetic radiation emitted by the emitter can be configured to form standing waves within the chamber, thereby ensuring a well-defined electromagnetic field structure of nodes and antinodes; in addition, the igniter may be configured to induce ionization exactly at an optimal field maximum. Such a spatially and temporally coordinated configurations of the emitter and igniter may minimize variability in plasma initiation, allowing stable and predictable plasma formation (plasma ignition). This allows for scaling the apparatus to larger dimensions or higher throughput applications. Because plasma initiation efficiency is significantly improved through a combined operation of the emitter and the igniter, the chamber can be scaled in size and shape more freely (columnar, cylindrical, or otherwise), enabling modular design.

As also explained in detail below, the combined operation of the emitter and the igniter allows for a broader scalability of the plasma and the chamber (i.e., reactor), thereby expanding its applicability for the purposes described herein. As a specific example, the chamber may be provided as a cylindrical chamber elongated in a longitudinal, height direction. The combined operation of the emitter and the igniter in the manner described herein may allow for the height to be configurable within a large range from 0.1 m to 10 m (or even larger), depending on the application and use case.

Further beneficial effects and advantages of the present invention and its embodiments are described in further detail below.

The atmospheric pressure as referred to herein may indicate an air pressure (barometric pressure) in a given environment on Earth without additional pressurization or evacuation (i.e., without modification of pressure). The atmospheric pressure may fluctuate around 1 atm (1 standard atmosphere) or 1,013.25 hPa by a deviation of up to 10% (or 5%) thereof, depending on, for example, the temperature and weather conditions. In this disclosure, the atmospheric conditions may include a temperature of about 20° C., unless indicated otherwise or technically inappropriate.

At atmospheric pressure, achieving plasma ignition typically requires overcoming relatively high breakdown thresholds, thus necessitating careful alignment and synchronization of the igniter's energy input with electromagnetic radiation conditions (e.g., field maxima of standing waves generated by the emitter). The combined operation of the emitter and the igniter ensures that minimal energy is consumed, enhances operational efficiency, reduces thermal stress on materials, and prevents undesirable thermal equilibrium states, maintaining plasma in a distinctly nonthermal (cold plasma) regime.

The term “gas discharge”, as used herein, refers to a physical process wherein a gas becomes electrically conductive as a result of ionization, allowing electric current to flow through the gas. Gas discharge is typically characterized by an electron density in a gas volume exceeding a specific threshold, as commonly understood in plasma physics, while maintaining overall electrostatic neutrality within that gas volume. Gas discharge is typically initiated by the application of energy, such as electrical, electromagnetic, or optical energy, sufficient to induce the ionization of the gas molecules.

The term “non-thermal plasma” as used herein, also referred to as cold plasma, denotes a plasma state in which the temperature of the electrons is one or more orders of magnitude higher than the temperature of the heavier species, such as ions and neutral gas molecules. These heavier species typically remain near ambient temperature, which gives rise to the term “cold.” Since the overall temperature of the gas volume is predominantly determined by the thermal energy of these heavier species, the bulk gas temperature does not rise significantly during plasma generation and maintenance. Accordingly, this type of plasma is referred to as nonthermal plasma, in contrast to thermal plasmas where all species are in thermal equilibrium at high temperatures.

In some examples, the chamber may have a columnar structure extending along a longitudinal axis. The chamber may comprise a sidewall extending around the longitudinal axis.

The chamber may have a columnar structure extending along a longitudinal axis, meaning that the chamber is elongated in one primary direction, forming a generally tubular or pillar-like geometry. The term “columnar” is to be interpreted broadly and includes shapes with circular, elliptical, polygonal, or irregular cross-sections. The longitudinal axis defines the central axis of elongation, along which at least one of gas flow, plasma formation, and energy propagation may be oriented or optimized. The chamber may comprise a sidewall extending around the longitudinal axis, where the sidewall forms the enclosing boundary of the chamber and defines the internal volume in which plasma is generated. The sidewall may be continuous, perforated, or segmented, and may be made of conductive or non-conductive material depending on its intended interaction with electromagnetic fields. For example, the chamber may be a cylindrical metal tube with a conductive sidewall surrounding a vertical axis, or a rectangular prism with a perforated polymer sidewall allowing controlled gas exchange. In some embodiments, the columnar shape facilitates the formation of standing electromagnetic waves or vertical airflow patterns, while the surrounding sidewall provides structural integrity, electromagnetic shielding, or gas permeability, depending on its construction.

In some examples, the chamber may have a substantially cylindrical geometry around the longitudinal axis and extends by a height of along the longitudinal axis.

The chamber may have a substantially cylindrical geometry around the longitudinal axis, meaning that the chamber has a shape that approximates a cylinder, with a generally circular or elliptical cross-section that is rotationally symmetric or nearly symmetric around the axis of elongation. The qualifier “substantially” is intended to include variations from an ideal cylinder, such as minor tapering, bulging, or polygonal faceting, which may arise from manufacturing constraints or functional adaptations. The chamber may extend by a height along the longitudinal axis, while the height may define a primary axial dimension of the chamber, which may correspond to a vertical dimension, depending on the orientation of the chamber. For example, the chamber may be a stainless-steel tube of cylindrical form with a height of 10 cm to 100 m, aligned vertically to support upward air convection and standing microwave field patterns. The cylindrical geometry may simplify field modeling and wave propagation, and may be well-suited for applications requiring standing electromagnetic wave and gas flow control.

In some examples, the sidewall of the chamber may be made of conductive material so as to shield an interior of the chamber from external electric fields.

In this context, the term “conductive material” may refer to an electrically conductive material in accordance with the teachings of the classical electrodynamics. For example, a conductive material may have an electrical conductivity of larger than 105 S/m at 20° C. Alternatively or additionally, a conductive material may be a material having a Fermi energy inside the conduction band (in terms of quantum mechanics). A conductive material may be or comprise a metal (e.g., stainless steel, aluminum, copper) or a conductive composite.

The expression “shield an interior of the chamber” may refer to a function of the conductive sidewall of the chamber as a Faraday shield (Faraday cage), which may prevent the penetration of external electromagnetic interference (EMI) into the interior of the chamber, i.e., into the plasma region. This shielding may ensure that the conditions within the chamber, particularly the electromagnetic field distribution required for plasma generation and maintenance, remain stable and unaffected by external noise or unintended fields. The conductive sidewall may be continuous or segmented and may also serve as a ground or reference potential surface in the overall electrical design. For example, the chamber may be formed as a seamless cylindrical tube made of stainless steel, with ports (perforations, holes, slits or the like) for gas inflow and electromagnetic coupling, thereby achieving effective shielding and field containment. Alternatively or additionally, the sidewall may comprise a conductive mesh or perforated metal sheet that both allows controlled gas exchange and provides partial electromagnetic shielding. This construction may be advantageous for maintaining standing electromagnetic waves as well as for maintaining precise plasma conditions.

In some examples, the sidewall of the chamber may be gas-permeable by being at least one of: at least partially perforated, partially discontinuous, or partially open.

The term “gas-permeable” may refer to a structural feature of the sidewall of the chamber that allows gas to enter into or exit from the chamber through its surface (i.e., through the sidewall), rather than solely through designated inlets or outlets. The permeability may enable passive or convective gas exchange, may support airflow during plasma operation, and may facilitate uniform gas replenishment in the chamber.

A “partially perforated” sidewall includes materials such as metal sheets, ceramics, or polymers with regularly or irregularly spaced holes or pores. A “partially discontinuous” sidewall refers to a structure composed of segments, panels, or lattices with intermediate gaps that break the continuity of the surface. A “partially open” sidewall may include designs such as a mesh grid or a framework with large open areas that allow gas flow. For example, the chamber may be formed as a cylindrical metal shell with perforated sections to admit ambient air radially into the interior of the chamber (i.e., to a volume surrounded by the sidewall of the chamber). In specific examples, the chamber may be made from a conductive wire mesh that provides both electromagnetic shielding and gas permeability. These features may be advantageous for applications involving ambient air processing, continuous gas flow, and self-sustaining convection within the plasma reactor.

In some examples, the igniter may comprise an electron source to generate free electrons at the ignition point by applying an electrical field.

An “electron source” as used herein may refer to a device or mechanism capable of emitting electrons into a gas volume when energized, typically through the application of a high-voltage or time-varying electric field. The emitted electrons may serve to initiate gas discharge by triggering ionization events that lead to the formation (ignition) of plasma. The electron source may be configured to apply an electrical field using at least one of, without being limited to: direct-current (DC) biasing, pulsed high-voltage sources, radio-frequency excitation, or inductive coupling. Specific examples of an electron source may include a spark gap, a high-voltage electrode pair, a thermionic emitter, and a Tesla coil. For instance, the igniter may include two needle-shaped electrodes separated by a small gap within the chamber, where a pulsed voltage is applied to produce a localized electron burst at the ignition point. In another example, a compact Tesla coil may be positioned externally to couple electrons inductively into the chamber through a dielectric window. This design may ensure reliable plasma initiation, particularly under atmospheric pressure, where a critical electron density must be achieved to overcome the ionization threshold of the gas.

In some examples, the chamber may be configured to support a standing wave of the electromagnetic radiation emitted by the emitter.

Accordingly, the geometry, arrangement and material properties of the chamber may be adapted such that electromagnetic waves emitted by the emitter into the chamber form standing wave patterns within the chamber. A standing wave may refer to a stationary oscillating field pattern resulting from the superposition of two or more waves of the same frequency traveling in opposite directions, typically as a result of wave reflection at an inner wall of the chamber. The expression “supporting a standing wave” may indicate that the chamber's internal dimensions (e.g., length, diameter) are commensurate with one or more half-wavelengths of the emitted electromagnetic radiation, forming resonant modes that amplify electric field intensities at specific locations (antinodes). These field maxima are particularly advantageous for efficient plasma generation, especially when aligned with the ignition point of the igniter.

For example, a cylindrical chamber with a length that corresponds to a multiple of approximately 11.5 cm may support a half-wavelength standing wave at 2.45 GHZ (standard microwave frequency), enabling field enhancement along the longitudinal axis. Alternatively, a rectangular or coaxial chamber with reflective end caps may be tuned to form transverse electric (TE) or transverse magnetic (TM) standing wave modes. Such resonant configurations may not only improve energy efficiency but also stabilize plasma characteristics by ensuring consistent field distributions within the chamber during operation.

In some examples, the emitter and the igniter may be configured such that the ignition point of the igniter approximately coincides (aligns) with a field maximum of the electromagnetic radiation emitted by the emitter.

Herein, a field maximum may refer to a spatial location, particularly within the chamber, where the electric field amplitude of the standing electromagnetic wave and/or electromagnetic radiation emitted by the emitter reaches its local or global peak. Coincidence, or alignment, of the ignition point of the igniter with such a field maximum may enhance the efficiency and reliability of plasma ignition. This is because the presence of a high electric field at the ignition point reduces the required ignition energy and increases the probability of ionizing collisions. The phrase “approximately coincides” allows for minor spatial deviations due to geometric tolerances or dynamic plasma effects but still implies a deliberate and functional spatial alignment.

For example, in a cylindrical microwave plasma chamber designed to support a standing wave at 2.45 GHZ, the ignition point may be positioned at an axial location corresponding to a quarter- or half-wavelength antinode, where the electric field amplitude is maximal. Alternatively, in a chamber with a loop antenna emitter, the igniter may be placed at the central hotspot of the generated field distribution. This coordinated positioning may enable efficient plasma formation at lower input power and supports stable operation at atmospheric pressure.

In some examples, the emitter may be configured to emit the electromagnetic radiation in a pulsed manner, in a continuous manner, or in an alternating combination of both.

Herein, emitting the electromagnetic radiation in a pulsed manner may refer to a generation of electromagnetic radiation in discrete bursts, characterized by a defined pulse duration, and optionally by a repetition rate and/or a duty cycle. Pulsed operation may be useful for controlling and reducing energy input, minimizing thermal load, or synchronizing with external ignition events.

Herein, emitting the electromagnetic radiation in a continuous manner may refer to an uninterrupted generation of electromagnetic radiation over time, typically used for maintaining stable plasma conditions once ignition has occurred.

Herein, emitting the electromagnetic radiation in an alternating combination of a pulsed and continuous manner may encompass operating modes where the emitter can switch between pulsed and continuous emission, either automatically or under external control, to adapt to varying operational conditions or process requirements. The emitter may be configured to alternate between pulses of electromagnetic radiation and continuous radiation. This flexibility in emission modes may support a wide range of plasma behaviors, including highly reactive transient discharges or sustained, stable nonthermal plasmas under atmospheric pressure.

In some examples, the emitter may be configured to emit (generate) the electromagnetic radiation in a wavelength range from 100 nanometers to 1 meter, or from 1 to 50 centimeters, or from 10 to 20 centimeters. In particular, the emitter may be configured to emit the electromagnetic radiation with a frequency of about 2.45 GHZ, which corresponds to a wavelength of about 12.23±0.05 centimeters, or to a wavelength from 12.20 to 12.25 centimeters.

The emitter may be configured to emit electromagnetic radiation in a wavelength range from 100 nanometers to 1 meter, which encompasses an electromagnetic spectrum including ultraviolet (UV), visible light, infrared (IR), microwave, and radio frequency (RF) bands. Within this range, the emitter may more specifically operate in narrower subranges, such as 1 to 50 centimeters, or even more particularly 10 to 20 centimeters, depending on the desired resonance conditions and the gas species being targeted. These wavelength ranges correspond to electromagnetic radiation frequencies suitable for inducing vibrational and rotational excitations in gas molecules, which is advantageous for efficient plasma generation. In particular, the emitter may be configured to emit radiation at a frequency of approximately 2.45 GHZ, corresponding to a wavelength of about 12.23±0.05 centimeters, or more precisely within a range of 12.20 to 12.25 centimeters. This frequency is widely used in industrial, scientific, and medical (ISM) applications and is particularly suitable for generating non-thermal plasma in air or gas mixtures, due to its well-established resonance characteristics with common atmospheric gases. For example, a magnetron operating at 2.45 GHz may be used to generate microwaves within a resonant chamber, enabling the formation of a stable standing wave and supporting efficient electron acceleration and ionization. Alternatively, a solid-state microwave generator may be tuned to this frequency range for precision-controlled plasma processes in modular or scalable reactor designs.

In some examples, the apparatus may further comprise a liquid injector configured to inject a liquid into the chamber. The liquid injector as used herein may also be referred to as a misting column, unless indicated otherwise or technically inappropriate. Moreover, the liquid injector may include any one, some or all of the features of the liquid injector or misting column as described below, unless indicated otherwise or technically inappropriate.

The term “liquid injector” used herein may refer to a component or system capable of delivering a controlled amount of liquid into the plasma chamber. The liquid injector may be configured to inject a liquid into the chamber either continuously or intermittently. The injection of the liquid by the liquid injector may be performed as droplets, jets, mist, spray, or vapor, depending on the design of the injector and the properties of the liquid. The liquid injector may operate under pressure, gravity, or capillary action, and may include nozzles, ultrasonic nebulizers, micropumps, or piezoelectric actuators to regulate the injection parameters such as flow rate, droplet size, and timing. The injector may be positioned radially, axially, or tangentially relative to the chamber geometry, and may be configured to operate synchronously with the emitter or igniter for timed chemical processing.

The liquid injected by the liquid injector may be or contain water. The liquid injected by the liquid injector may be selected so as to participate in plasma-induced chemical reactions, particularly in conversion of CO2 into methanol in the presence of water. Additionally or alternatively, the liquid injected by the liquid injector may be selected so as to serve controlling temperature, generating reactive species, or conditioning the plasma environment. For example, in one embodiment, the liquid injector may introduce water in the form of fine mist into the plasma zone, where water molecules are dissociated to form hydroxyl radicals or hydrogen, that may promote hydrogenation reactions of carbon dioxide stepwise to methanol.

In some examples, the chamber may comprise an outlet configured to discharge gas stream from the chamber.

The term “outlet” may refer to any opening, port, or conduit that allows gas, processed or unprocessed, to exit the chamber in a controlled manner. The “gas stream” may include at least one of: unreacted carrier gases (including air), plasma-activated species, reaction products, and byproducts, radicals, or trace compounds. The outlet may be positioned at one end of the chamber, e.g., axially aligned with the longitudinal axis, or laterally along the sidewall, depending on the desired gas flow profile.

The outlet may further comprise one or more flow-regulating elements such as a valve, a nozzle, a diffuser, a pump or a fan (blower) to control the discharge rate, direction, and pressure of the gas stream. In specific examples, the outlet may be part of a continuous flow system where ambient air or a process gas enters through inlets and/or gas-permeable sidewall(s) of the chamber and exits the chamber through the outlet. For example, in a vertical columnar chamber, the outlet may be located at the top to allow heated or chemically modified gases to exit via buoyant flow.

In some examples, the apparatus may further comprise a filter disposed at the outlet of the chamber and configured to capture at least one of: carbon oxides, nitrogen oxides, and sulfur oxides.

Herein, the term “filter” may be or comprise a physical, chemical and/or catalytic component positioned downstream of the volume where the nonthermal plasma is generated and sustained, for example at or near the outlet of the chamber. The filter may be configured to selectively remove or convert undesired species before they are released into the environment or passed to a subsequent process stage. The filter may operate through adsorption, absorption, condensation, catalytic conversion, or a combination thereof. The targeted substances may include at least one of: carbon oxides (e.g., CO, CO₂), nitrogen oxides (e.g., NO, NO₂), or sulfur oxides (e.g., SO₂). For example, the filter may comprise activated carbon, zeolite, or a catalytic ceramic substrate coated with transition metals to promote oxidation or reduction reactions. The placement of the filter at the outlet may ensure that plasma-processed gas streams are purified before emission or recirculation,

The term filter may be used herein in a collective manner also for an air filter or a carbon filter. The filter as used herein may also be referred to as an air filter or a carbon filter, unless indicated otherwise or technically inappropriate. Moreover, the filter may include any one, some or all of the features of the air filter or carbon filter as described below, unless indicated otherwise or technically inappropriate.

In some examples, the apparatus may further comprise a water tank in fluid communication with the chamber and configured to dissolve methanol from gas stream discharged from the chamber.

The water tank as used herein may refer to a container or reservoir holding water or an aqueous solution, which is connected, for example directly or via tubing, piping, or fluid conduits, to (the outlet of) the plasma chamber. The phrase “in fluid communication” may denote that gas discharged from the chamber can be directed into contact with the water contained in the tank, either by bubbling through, diffusing into, or being sprayed over the liquid surface. The water tank may be configured to absorb methanol vapor or droplets present in the gas stream into the water phase, based on methanol's high solubility in water. This feature may enable the collection and partial separation of methanol from other gaseous components, such as CO2, unreacted air, or radicals. For example, the gas stream containing methanol formed by plasma-induced reactions between air and injected water may be directed through a sparger or gas diffuser submerged in the water tank, allowing methanol to dissolve while residual gases are vented or sent for further treatment. The water tank as used herein may include any one, some or all of the features of the water tank as described below, unless indicated otherwise or technically inappropriate.

According to an aspect, a system is provided. The system may comprise an air chamber system, a nonthermal plasma generation unit and a fluid injector. The air chamber system may comprise a sidewall made of a conductive material and surrounding a longitudinal axis, the air chamber system being configured to be permeable to gas. The nonthermal plasma generation unit may be configured to generate nonthermal plasma within a volume surrounded by the sidewall of the air chamber system by means of an electromagnetic radiation in a wavelength range of 100 nanometers to 1 meter and an igniter configured to induce gas discharge. The fluid injector may be configured to introduce a liquid into the volume surrounded by the sidewall of the air chamber system.

The system may allow for plasma-assisted processing of an ambient gas, particularly ambient air, under ambient conditions. The system may be capable of generating and sustaining nonthermal plasma in a controllable and scalable manner.

The air chamber system, with a sidewall made of conductive material, may provide structural integrity and may act as an electromagnetic shield, suppressing interference from external fields and containing the electromagnetic energy within the plasma volume. The air chamber system may be permeable to gases through perforations, gaps, or porous construction and may allow for passive or convective gas exchange. This may support continuous or semi-continuous flow-through operation.

The nonthermal plasma generation unit may comprise an electromagnetic emitter (e.g., microwave source operating at 2.45 GHz) and an igniter (e.g., electrode, spark gap, or electron source). The nonthermal plasma generation unit may enable localized plasma initiation via gas discharge and efficient energy transfer into a larger volume within the air chamber system without significant heating. This may result in a chemically active yet thermally benign environment that is ideal for processing sensitive molecules or ambient air components.

The inclusion of a fluid injector may allow for the introduction of reactive liquids (e.g., water or methanol) directly into the plasma zone, enabling plasma-enhanced reactions such as CO2 reduction, pollutant degradation, or methanol generation.

Accordingly, a compact system is provided capable of utilizing nonthermal plasma for environmental purposes at atmospheric pressure, with high energy efficiency, enlarged scalability, and suitability for environmental, chemical, or energy-related applications.

The system as referred to herein may comprise, or implemented by or correspond to, the apparatus as disclosed herein, unless indicated otherwise or technically inappropriate. The system may incorporate any one, some or all of the features of any of the apparatuses disclosed herein, unless indicated otherwise or technically inappropriate. Furthermore, the system may incorporate any one, some or all of the features of any of the methods disclosed herein, unless indicated otherwise or technically inappropriate.

The air chamber system of the system may be or comprise the chamber of the apparatus as described herein. Accordingly, the air chamber system may incorporate any one, some or all of the features of the chamber disclosed herein, unless indicated otherwise or technically inappropriate.

The nonthermal plasma generation unit of the system may comprise, or implemented by or correspond to, a combination of the emitter and the igniter as disclosed herein. Accordingly, the nonthermal plasma generation unit may incorporate any one, some or all of the features of the emitter and the igniter as disclosed herein, unless indicated otherwise or technically inappropriate. Similarly, the emitter and the igniter may, alone or in combination, incorporate any one, some or all of the features of the nonthermal plasma generation as disclosed herein, unless indicated otherwise or technically inappropriate. For example, the volume surrounded by the sidewall may correspond to a volume in which the emitter and igniter are operable in conjunction to generate and maintain nonthermal plasma under atmospheric conditions.

The fluid injector may comprise, or implemented by or correspond to, the liquid injector as described herein. Similarly, liquid injector may comprise, or implemented by or correspond to, the fluid injector as described herein.

According to an aspect, a method of plasma-assisted processing of air is provided. The method may comprise generating nonthermal plasma in a chamber at atmospheric pressure, the chamber being configured to permit airflow through the chamber. the method may comprise injecting water into the chamber while maintaining the nonthermal plasma in the chamber and permitting airflow through the chamber.

The method may allow for conversion of gas species, such as carbon compounds or pollutants, from ambient air, using a nonthermal plasma reactor operating under atmospheric pressure. The method may incorporate any one, some or all of the features of any of the apparatuses or systems disclosed herein, unless indicated otherwise or technically inappropriate. The method may incorporate any one, some or all of the features of any other method disclosed herein, unless indicated otherwise or technically inappropriate.

The step of generating nonthermal plasma in a chamber may provide a chemically active environment rich in energetic electrons, ions, radicals, and excited species, while maintaining the overall gas temperature near ambient levels. This may promote the activation or conversion of stable atmospheric molecules (e.g., CO2, N2, O2, etc.) without the need for high-temperature processes. The chamber may be specifically configured to permit airflow, meaning air can pass through or across the plasma region, enabling continuous or semi-continuous treatment of air streams. This airflow may ensure replenishment of reactants and removal of reaction products, supporting steady-state operation. The chamber as mentioned in the method may correspond to the chamber of the apparatus or system as described herein.

The additional step of injecting water into the chamber may introduce a source of hydrogen and hydroxyl radicals when exposed to the plasma, which may promote hydrogenation reactions of carbon dioxide to methanol.

For example, plasma-induced dissociation of water may generate hydrogen that reacts with CO2 from the air to form syngas or methanol precursors. The co-occurrence of water injection and plasma excitation in a flowing air environment may lead to enhanced extraction, conversion, or breakdown of substances such as CO2, volatile organic compounds, or nitrogen oxides. The technical effects of the method include high selectivity and energy efficiency, scalability for atmospheric applications, minimal thermal footprint, and compatibility with open-air or inline gas-processing systems, making it suitable for environmental remediation, air purification, or green chemical synthesis.

According to an aspect, a method for synthesis of methanol from a gas that contains carbon dioxide is disclosed. The method may comprise generating nonthermal plasma in a chamber. The method may comprise inducing dissociation of CO2 from the gas to CO and O by introducing the gas into the chamber while sustaining the nonthermal plasma in the chamber. The method may comprise introducing water into the chamber, thereby inducing hydrogenation of CO to methanol.

The method may incorporate any one, some or all of the features of any of the apparatuses or systems, or any other method disclosed herein, unless indicated otherwise or technically inappropriate. Same terms may refer to the same or like components as described herein.

In some examples, the nonthermal plasma may be generated at atmospheric pressure. In some examples, the chamber may comprise a conductive sidewall that shields the plasma from external electric field.

In some examples, the nonthermal plasma may be generated by introducing electromagnetic radiation in a wavelength range of 100 nanometers to 1 meter into the chamber and at the same time providing energy at an ignition point within the chamber to initiate a gas discharge.

In some examples, the nonthermal plasma may be generated by introducing electromagnetic radiation in a microwave frequency range, for example at a frequency of about 2.45 GHz.

In some examples, the nonthermal plasma may be sustained in a standing wave mode of the electromagnetic radiation within the chamber.

In some examples, the water may be introduced in the form of a mist, spray, or fine droplets into the nonthermal plasma within the chamber.

In some examples, the method may further comprise dissolving methanol from the gas stream exiting the chamber in a water tank.

In some examples, the gas that contains carbon dioxide may be or comprise ambient air.

According to an aspect, a method for synthesis of methanol from a gas that contains carbon dioxide is disclosed. The method may comprise generating nonthermal plasma in a chamber using electromagnetic radiation in combination with an electron source. The method may comprise introducing water droplets and a gas containing carbon dioxide into the chamber while sustaining the nonthermal plasma in the chamber.

The method may incorporate any one, some or all of the features of any of the apparatuses or systems, or any other method disclosed herein, unless indicated otherwise or technically inappropriate. Same terms may refer to the same or like components as described herein.

According to an aspect, an apparatus configured for synthesis of methanol out of a gas that contains carbon dioxide is disclosed. The apparatus may comprise an air chamber system, a nonthermal plasma generation unit and a fluid injector. The air chamber system may be configured to sustain nonthermal plasma within a volume. The nonthermal plasma generation unit may be configured to generate nonthermal plasma in the volume. The fluid injector configured to introduce water into the volume.

The apparatus may incorporate any one, some or all of the features of any other apparatus or any of the systems, or any of the methods disclosed herein, unless indicated otherwise or technically inappropriate. Same terms may refer to the same or like components as described herein.

In some examples, the chamber may comprise a sidewall surrounding the volume, wherein the sidewall is at least partly permeable to gases.

In some examples, the nonthermal plasma generation unit may comprise an emitter configured to generate electromagnetic radiation in a wavelength range from 100 nanometers to 1 meter.

In some examples, the fluid injector may be configured to introduce water into the volume as droplets or mist.

In some examples, the apparatus may further comprise a water tank connected downstream of the air chamber system.

Accordingly, a scalable apparatus and method for addressing elevated CO2 levels in the atmosphere is presented. This innovative system utilizes nonthermal plasma to convert Carbon Dioxide (CO2) into Carbon Monoxide (CO) and Oxygen. In addition to CO2 removal, the disclosed technology further integrates the synthesis of green fuel (e.g., methanol) through the introduction of microdroplets of water.

In preferred embodiments, the apparatus is designed with cost-effectiveness in mind, featuring a controller, power management unit, nonthermal plasma chamber for CO2 reduction and green fuel (e.g., methanol) production. The nonthermal chamber comprises a pulsed high-frequency directional electromagnetic transmitter, high-voltage electrodes, a misting column, a faraday cage and a vortex-enabled gas chamber.

A significant aspect of this technology is the incorporation of green fuel (e.g., methanol) production, achieved by introducing microdroplets of water into the system. The process involves the interaction of water droplets with the converted CO in the nonthermal plasma chamber, leading to the synthesis of green fuel (e.g., methanol). This method provides an environmentally friendly and sustainable approach to fuel production without the reliance on traditional chemical processes.

Another significant aspect of this technology is the directly utilization of CO2 from diluted sources like flue gas and ambient air by taking advantage of the reaction selectivity of nonthermal plasma. This method eliminates the need of carbon capturing and purifying by directly converting CO2 into other product like CO or green fuel (e.g., methanol).

Another significant aspect of this technology is the directly synthesis of higher carbon fuel like green diesel or sustainable aviation fuel by integrating an additional catalyst to convert the generated green fuel (e.g., methanol) with one additional step.

The presented innovation represents a purely physical method to mitigate CO2 levels without the use of chemicals or consumables, making it a holistic solution for atmospheric/flue gas carbon reduction and green fuel synthesis.

The present invention pertains to configuration and operation of an apparatus for generating and maintaining an atmospheric plasma. the field of atmospheric/flue gas carbon reduction technologies and, more specifically, introduces a physical, scalable, and cost-effective approach for mitigating CO2 levels. This innovation can be implemented across various embodiments, showcasing adaptability and versatility in its application.

The focus of this invention is on a CO2 reduction method that distinguishes itself by its reliance on purely physical processes, omitting the need for chemical interventions. This feature not only ensures a more environmentally friendly operation but also contributes to energy efficiency by eliminating high-energy-consuming chemical processes traditionally associated with carbon capture technologies.

A key aspect of the invention involves the seamless integration of nonthermal plasma technology into a singular compact apparatus. By doing so, the disclosed system offers a direct and efficient means of removing CO2 directly from the atmosphere. This integration streamlines the overall operation, making it an attractive solution for deployment in various environmental and industrial settings.

The scalable nature of the invention further enhances its adaptability, enabling deployment in diverse scenarios ranging from industrial applications to environmental restoration projects. The low-cost elements incorporated into the design, including a controller, power management unit, and nonthermal plasma chamber, contribute to the economic feasibility of widespread implementation.

In summary, the present invention represents a groundbreaking development in the field of carbon reduction technologies, providing a unique and comprehensive solution that is both scalable and cost-effective. The integration of nonthermal plasma into a compact apparatus ensures a direct and efficient removal of CO2 from the atmosphere/flue gas, while simultaneously enabling the production of green fuel like methanol—a key component in the transition towards a sustainable and carbon-neutral future. This distinguishes it as a pioneering technology with far-reaching implications for environmental sustainability and renewable energy initiatives.

Carbon Dioxide (CO2) stands as the primary greenhouse gas responsible for global warming, with its concentration in the atmosphere witnessing a 50% increase over the past 200 years due to human activities.

The stark reality of climate disruption became apparent in the Northern Hemisphere during the summer of 2022. The Intergovernmental Panel on Climate Change (IPCC) projects the urgent need to remove 10 gigatons of CO2 annually by 2050, a number that surges to 40 gigatons by 2100.

Recent reports underscore the urgency of addressing climate change, indicating the need to remove significant quantities of CO2 even before 2050, far exceeding previous projections.

Unlike current CO2 capturing techniques, the presented invention adopts a unique approach by leveraging nonthermal plasma for the direct conversion of CO2 into green fuel like Methanol, eliminating the need for chemical additives. The absence of carbon capture and electrolysis costs distinguishes this method, providing an economically viable and sustainable solution.

The technology in question stands apart by selectively breaking down CO2 into CO, requiring less energy and eliminating the need for additional carbon capturing costs. This approach contrasts sharply with existing methods, offering a more efficient and cost-effective pathway for atmospheric/flue gas CO2 removal and/or utilization.

The innovation extends beyond CO2 removal to encompass the synthesis of green fuel like methanol. By introducing microdroplets of water into the system, the technology facilitates the interaction with converted CO, resulting in the sustainable production of green fuel like methanol. This environmentally friendly approach to green fuel production (e.g., methanol) stands as a valuable contribution to the sustainable energy landscape.

The present invention distinguishes itself by scaling up nonthermal plasma to achieve stepwise vibrational excitation, requiring less energy for CO2 bond breaking without the need of using pure CO2.

While it suggests higher operating frequencies for improved efficiency, the present invention achieves better efficiency at normal atmospheric pressure, reducing energy requirements and operational costs. The present invention distinguishes itself by being able to work directly with diluted source of CO2 while achieving better efficiency.

In summary, the present invention represents a breakthrough in CO2 reduction and green fuel (e.g., methanol) synthesis, offering an energy-efficient, cost-effective, and scalable solution that aligns with the urgent IPCC climate goals. The unique application of nonthermal plasma sets this technology apart, paving the way for a sustainable and impactful approach to atmospheric/flue gas carbon management, coupled with the synthesis of green fuel (e.g., methanol) for a comprehensive contribution to the global transition towards renewable energy.

The nonthermal plasma system and state-of-the-art CO2 removal and utilization method herein disclosed offer a direct utilization of CO2 concentration at atmospheric pressure. This scalable method provides an effective solution to global warming and aligns with the urgent needs outlined by the Intergovernmental Panel on Climate Change (IPCC) to remove 10 gigatons of CO2 from the atmosphere annually by 2050. Additionally, the synthesis of green fuel (e.g., methanol) through this process further contributes to mitigating climate change. The low-energy, compact, and cost-effective apparatus utilizing nonthermal plasma is disclosed, wherein the plasma is generated internally, distinguishing it from other methods of capturing and breaking down CO2. The CO2 removal and utilization system herein disclosed comprises a nonthermal plasma generation unit, air chamber system, faraday cage, misting column, measurement sensors, data storage unit, and power unit. Notably, this system features high efficiency and relatively low power requirements compared to conventional CO2 removal and utilizations techniques that rely on bulky fans, chemical elements, high temperatures, high pressures and suffer from limited throughput.

It is therefore an object of the present invention to disclose a scalable, compact and low-cost CO2 removal and utilization apparatus does not have recourse to chemical processes, such as catalysis for its basic operation of green fuel (e.g., methanol) synthesis. In its preferred embodiment comprises of (a) an air chamber system that works under atmospheric pressure, (b) a nonthermal plasma generation unit; (c) a faraday cage; (d) a misting column; (e) measurement, communication, storage and power units.

It is a further object of this invention to disclose a scalable, compact and low-cost CO2 removal, capability wherein said it breaks CO2 into CO+O.

It is a further object of this invention to disclose a scalable, compact and low-cost green fuel synthesis (e.g., methanol).

It is therefore an object of the present invention to disclose an air chamber system that in its preferred embodiment comprises of a cylindrical shape tube aligned vertically to fit on the top of the nonthermal plasma generation unit.

It is a further object of this invention to disclose such of a cylindrical shape tube as described in any of the above, further comprises of multiple long vertical slits that act as an inlet for ambient air for direct air utilization mode. These slits redirect air to sustain a vortex flow inside the air chamber system.

It is a further object of this invention to disclose such of a cylindrical shape tube as described in any of the above, further comprises of sealed chamber with an inlet for direct flue gas utilization mode.

It is a further object of this invention to disclose such of a cylindrical shape tube as described in any of the above, wherein air accelerates its speed inside the chamber due to the vortex effect which would result in an air/plasma whirlwind.

It is a further object of this invention to disclose such of a cylindrical shape tube as described in any of the above, wherein air would have a different pressure inside the tube due to its increasing air speed which would make a pressure difference with the surrounding atmosphere or with the flue gas source allowing more air to be accelerated towards the inlets. That would eliminate the need of bulky fans to suck air/flue gas into the chamber.

It is a further object of this invention to disclose such of a cylindrical shape tube as described in any of the above, wherein circulating air is kept longer inside the chamber.

It is a further object of this invention to disclose such of a cylindrical shape tube as described in any of the above, further comprises of an outlet exit at the top end of the cylinder where result gases exit in a vortex shape and passed to the atmosphere or to collection and filtration tanks due to gained energy after being processed by the low energy nonthermal plasma generation unit.

It is a further object of this invention to disclose such of a cylindrical shape tube as described in any of the above, wherein the air column extends above the outlet exit achieving more effective area comparing to apparatus size.

It is a further object of this invention to disclose such of a cylindrical shape tube as described in any of the above, has an internal faraday cage tube.

It is therefore an object of the present invention to disclose a low energy nonthermal plasma generation unit, that in its preferred embodiment comprises of (a) high frequency directed electromagnetic beam and, (b) an electron source.

It is a further object of this invention to disclose such of a low energy nonthermal plasma generation unit as described in any of the above, that in its preferred embodiment, the high frequency directed electromagnetic beam is a pulsed microwave emitter (e.g., magnetron) that has a connected waveguide and antenna at the top, bottom or side end of the air chamber.

It is a further object of this invention to disclose such of a low energy nonthermal plasma generation unit as described in any of the above, that in its preferred embodiment, the pulsed microwave emitter has a waveguide that amplifies and directs the generated microwave towards the air chamber.

It is a further object of this invention to disclose such of a low energy nonthermal plasma generation unit as described in any of the above, that in its preferred embodiment, the pulsed microwave emitter waveguide and antenna allow standing waves to form inside the air chamber.

It is a further object of this invention to disclose such of a low energy nonthermal plasma generation unit as described in any of the above, that in its preferred embodiment, the pulsed electromagnetic emitter has a frequency, for example 1.3 GHZ, and has harmonics that matches the resonance frequency of the air/flue gas N2 or one of its based chemical compounds and/or CO2 bonds which allow the later to be excited with minimum possible power.

It is a further object of this invention to disclose such of a low energy nonthermal plasma generation unit as described in any of the above, wherein the pulsed electromagnetic emitter power and the waveguide size are proportional to the air chamber size.

It is a further object of this invention to disclose such of a low energy nonthermal plasma generation unit as described in any of the above, that in its preferred embodiment, the electron source is a pair of high voltage electrodes that is fitted over the waveguide exit of the high frequency directed electromagnetic beam generation unit.

It is a further object of this invention to disclose such of a low energy nonthermal plasma generation unit as described in any of the above, wherein the high voltage electrodes pair is separated by an insulator (e.g., air gap).

It is a further object of this invention to disclose such of a low energy nonthermal plasma generation unit as described in any of the above, wherein the high voltage electrodes pair ignites a plasma in the insulator by applying constant or alternating high voltage to it.

It is a further object of this invention to disclose such of a low energy nonthermal plasma generation unit as described in any of the above, wherein the pulsed electromagnetic emitter generates high frequency (e.g., microwave) radiation inside the air chamber which increases the energy state of the gas molecules.

It is a further object of this invention to disclose such of a low energy nonthermal plasma generation unit as described in any of the above, wherein the high voltage sparks generated by the electrodes pair in the insulator (e.g. air gap) meet standing waves node of the electromagnetic radiation inside the air chamber.

It is a further object of this invention to disclose such of a low energy nonthermal plasma generation unit as described in any of the above, wherein the generated plasma by the high voltage electrodes pair will expand to ionize most of the gas molecules inside the air chamber due to avalanche ionization and multiphoton ionization along with potential air vortex movement.

It is a further object of this invention to disclose such of a low energy nonthermal plasma generation unit as described in any of the above, wherein the short high frequency electromagnetic pulse vibrates N2 or one of its compounds molecules which transfer their vibrations to CO2 bonds to the maximum which result in breaking it into CO+O instantly in a stepwise vibrational excitation along with other electrons in plasma, requiring less energy compared to doing the same process with heat, or in thermal equilibrium.

It is a further object of this invention to disclose such of a low energy nonthermal plasma generation unit as described in any of the above, wherein excited N2 (or one of its compound molecules like NxOy) and/or CO2 molecules will make collisions other unexcited CO2 molecules in the chamber causing the first to lose their gained energy in vibrational-translational (VT) relaxation which would depopulates the vibrational levels of CO2 that would result in an energy-efficient CO2 conversion into CO+O by the vibrational ladder climbing pathway.

It is a further object of this invention to disclose such of a low energy nonthermal plasma generation unit as described in any of the above, wherein CO2 conversion efficiency into CO+O would increase as more collisions happen inside the air chamber which speeds up the vibrational depopulation process.

It is a further object of this invention to disclose such of a low energy nonthermal plasma generation unit as described in any of the above, wherein the broken CO and O will hit other CO2 bonds in the chamber causing a chain reaction that increases the efficiency of process inside the air chamber.

It is a further object of this invention to disclose such of a low energy nonthermal plasma generation unit as described in any of the above, wherein the short electromagnetic (e.g., microwave) pulse doesn't give a chance for CO to gain enough energy to bond again with monatomic oxygen.

It is a further object of this invention to disclose such a scalable, compact and low-cost CO2 removal apparatus as described in any of the above, further comprises of a misting column that consists of one or more water microdroplets source.

In the context of this invention, the term ‘microdroplets’ is hereby defined as minute liquid particles of water, typically ranging in size from approximately 1 to 100 micrometers in diameter. Microdroplets possess distinct physical characteristics from water vapor, being in a condensed liquid phase rather than a gaseous state. Notably, microdroplets exhibit higher surface area-to-volume ratios compared to larger liquid bodies, facilitating efficient interaction with gaseous constituents such as CO2 within the cold plasma reactor environment.

It is essential to distinguish microdroplets from other forms of water, including but not limited to bulk liquid water and water vapor. Bulk liquid water typically exists in larger volumes and lacks the fine dispersion characteristic of microdroplets. Water vapor, on the other hand, consists of individual water molecules in a gaseous state and does not exhibit the cohesive liquid properties inherent to microdroplets.

In the realm of cold plasma technology, microdroplets offer distinct advantages over water vapor due to their condensed liquid phase. Unlike water vapor, which consists of individual water molecules in a gaseous state, microdroplets exhibit cohesive properties inherent to liquids, allowing them to maintain higher densities and surface tension. This characteristic facilitates improved interaction with gaseous constituents, such as CO2, within the cold plasma reactor environment. Additionally, the condensed nature of microdroplets ensures a higher surface area-to-volume ratio, enhancing the efficacy of surface interactions and mass transfer processes. Consequently, the utilization of microdroplets in the cold plasma reactor system leads to more efficient CO2 utilization and removal, ultimately contributing to enhanced reactor performance and operational efficiency.

In the specific context of the disclosed cold plasma reactor system, the inclusion of a misting column comprising one or more water microdroplet sources serves as a pivotal component for achieving scalable, compact, and cost-effective CO2 removal. The utilization of microdroplets facilitates enhanced surface interaction and mass transfer between the water phase and gaseous CO₂ constituents within the reactor chamber, thereby optimizing the efficiency of CO2 removal and utilization processes. Furthermore, the integration of microdroplet technology enables precise control over reactor performance parameters, ensuring optimal operating conditions for CO2 removal and utilization while minimizing energy consumption and operational costs.

It is a further object of this invention to disclose such of a misting column as described in any of the above, wherein the water microdroplets units are made of high frequency (e.g., ultrasonic) atomizing units.

It is a further object of this invention to disclose such of a misting column as described in any of the above, wherein the water microdroplets units send pulses of water misting into the air chamber just after CO2 molecules get broken into CO+O.

It is a further object of this invention to disclose such of a low energy nonthermal plasma generation unit as described in any of the above, wherein another generated pulse plasma by the high voltage electrodes pair will expand to ionize most of the gas and water molecules inside the air chamber due to avalanche/multiphoton ionization and potential air vortex movement.

It is a further object of this invention to disclose such of a low energy nonthermal plasma generation unit as described in any of the above, wherein H2O conversion efficiency into H+OH would increase as more collisions happen inside the air chamber which speeds up the vibrational depopulation process.

It is a further object of this invention to disclose such of a low energy nonthermal plasma generation unit as described in any of the above, wherein the broken CO and H will combine to form methanol molecules, CH3OH.

It is a further object of this invention to disclose an internal faraday cage tube as described in any of the above, where in this faraday cage tube is made of metal to shield electromagnetic waves generated from the low energy nonthermal plasma generation unit.

It is a further object of this invention to disclose such of a faraday cage tube as described in any of the above, where in this faraday cage tube allows electromagnetic standing waves to be formed inside.

It is a further object of this invention to disclose such of a faraday cage tube as described in any of the above, where in this faraday cage tube amplifies the electromagnetic waves and its standing waves nodes.

It is a further object of this invention to disclose such of a faraday cage tube as described in any of the above, where in this faraday cage tube material can be made or coated with one or more material to act as an in-catalyst, pre-catalyst or post catalyst for plasma.

It is a further object of this invention to disclose such of a faraday cage tube as described in any of the above, where in this faraday cage tube material increases the efficiency or breaking CO2 molecules and generating green fuel (e.g., methanol).

It is a further object of this invention to disclose such of a faraday cage tube as described in any of the above, where in this faraday cage tube has small air slits spread all over its sides to allow air to get in for atmospheric CO2 utilization.

It is a further object of this invention to disclose such of a faraday cage tube as described in any of the above, where in these air slits don't block the air flow and vortex.

It is a further object of this invention to disclose such of a faraday cage tube as described in any of the above, where in these air slits don't block the water microdroplets flow generated by the misting column.

It is a further object of this invention to disclose such of a faraday cage tube as described in any of the above, where in these air slits have small diameter that is less than ¼ the wavelength of the fundamental electromagnetic waves' frequency generated inside to block it from going out.

It is a further object of this invention to disclose such of a faraday cage tube as described in any of the above, where in these air slits have small diameter that is less than ¼ the wavelength of the electromagnetic waves' frequency harmonics generated inside to block them from going out.

It is a further object of this invention to disclose such a scalable, compact and low-cost CO2 removal and utilization apparatus, further connects its top outlet to a pipe leading to the bottom of a tank (e.g., water tank).

It is a further object of this invention to disclose such a water tank collected the generated green fuel (e.g., methanol) by dissolving them into water while leaving other gases to leave.

It is a further object of this invention to disclose such a scalable, compact and low-cost CO2 removal and utilization apparatus as described in any of the above, further comprises of a controller to modulate the frequencies of the nonthermal plasma generation unit and misting column, manage measurements sensors and monitor the power.

It is a further object of this invention to disclose such a scalable, compact and low-cost CO2 removal and utilization apparatus as described in any of the above, further comprises of a measurement sensor that measures the plasma properties inside the air chamber.

It is a further object of this invention to disclose such a scalable, compact and low-cost CO2 removal apparatus as described in any of the above, further comprises of a measurement sensor that measures forward and reflected power for the nonthermal plasma generation unit along with the electron source.

It is a further object of this invention to disclose such a scalable, compact and low-cost CO2 removal and utilization apparatus as described in any of the above, discloses a method to monitor the amount of CO2 being processed by monitoring the measurement sensor readings for impedance and power.

It is a further object of this invention to disclose such a scalable, compact and low-cost CO2 removal and utilization apparatus as described in any of the above, further comprises of measurement sensors that monitor the CO, H, H2O and CO2 level in the surrounding atmosphere wherein the readings are used as feedback for the nonthermal plasma generation unit power.

It is a further object of this invention to disclose such a scalable, compact and low-cost CO2 removal and utilization apparatus as described in any of the above, further comprises of an optical measurement sensor (e.g., spectrometer) that monitor the ratio of different gases inside the air chamber and the water tank.

It is a further object of this invention to disclose such a scalable, compact and low-cost CO2 removal and utilization apparatus as described in any of the above, further comprises of wireless communication systems.

It is a further object of this invention to disclose such a scalable, compact and low-cost CO2 removal and utilization apparatus and utilization, wherein said wireless communication systems communicate with a communication network.

It is a further object of this invention to disclose such a scalable, compact and low-cost CO2 removal and utilization apparatus, further connects to blockchain service on clouds that updates the information and accessible by said communication network.

It is a further object of this invention to disclose such a scalable, compact and low-cost CO2 removal and utilization apparatus, wherein said blockchain service is constantly updatable in real time.

It is a further object of this invention to disclose such a scalable, compact and low-cost CO2 removal apparatus as defined in any of the above, further comprises of a local digital signal processor (DSP).

It is a further object of this invention to disclose such a scalable, compact and low-cost CO2 removal and utilization apparatus, wherein said local DSP is programmed along with one measurement sensor to carry out at least one appropriate algorithm to measure the impedance of the nonthermal plasma inside the air chamber system which can be used to measure the weight in kilograms of CO2 removed and utilized.

It is a further object of this invention to disclose such a scalable, compact and low-cost CO2 removal and utilization apparatus, wherein said local DSP performs a local blockchain validation.

It is a further object of this invention to disclose such a scalable, compact and low-cost CO2 removal and utilization apparatus as defined in any of the above, further comprises of a local memory.

It is a further object of this invention to disclose such a scalable, compact and low-cost CO2 removal and utilization apparatus, wherein said local memory can be a fixed memory or a volatile memory.

It is a further object of this invention to disclose such a scalable, compact and low-cost CO2 removal and utilization apparatus, further comprises of a database of blockchain entities stored in said local memory.

It is a further object of this invention to disclose such a scalable, compact and low-cost CO2 removal and utilization apparatus, wherein said the stored information is a combination of serial number, identity data, date and time, location, sensor readings and CO2 quantified processed data. All hashed together as a blockchain entity.

It is a further object of this invention to disclose such a scalable, compact and low-cost CO2 removal and utilization apparatus, wherein said the publicly available blockchain technology is providing a high value foundation for a digital green currency based on CO2 removal amounts.

It is a further object of this invention to disclose such a scalable, compact and low-cost CO2 removal and utilization apparatus, wherein said the power unit can be connected to a sustainable energy source like solar panel.

### BRIEF DESCRIPTION OF DRAWINGS

The invention will herein be described with reference to the drawings, wherein:

FIG. 1 illustrates a schematic diagram of the nonthermal plasma apparatus system layout of a possible and non-restricting embodiment in the scalable CO2 removal/utilization and green fuel (e.g., methanol) synthesis system herein disclosed; FIG. 1a and FIG. 1b show a close-up view to the top section of the system. FIG. 1c shows a close-up view to the misting column and misting units.

FIG. 2 illustrates schematic diagrams of vortex-enabled air chamber system layout of a possible and non-restricting embodiment in the nonthermal plasma apparatus system herein disclosed;

FIG. 3 illustrates schematic diagrams of a possible and non-restricting embodiment of green fuel (e.g., methanol) collection system herein disclosed; FIG. 3a shows a close-up view to the outlet interface.

### DETAILED DESCRIPTION

The following descriptive items detail the invention according to the accompanying figures. However, the invention is unlimited to the following listed clauses, but only as narrowed in the following claims, considering the broadest appropriate scope determined by the deepest understanding of said claims.

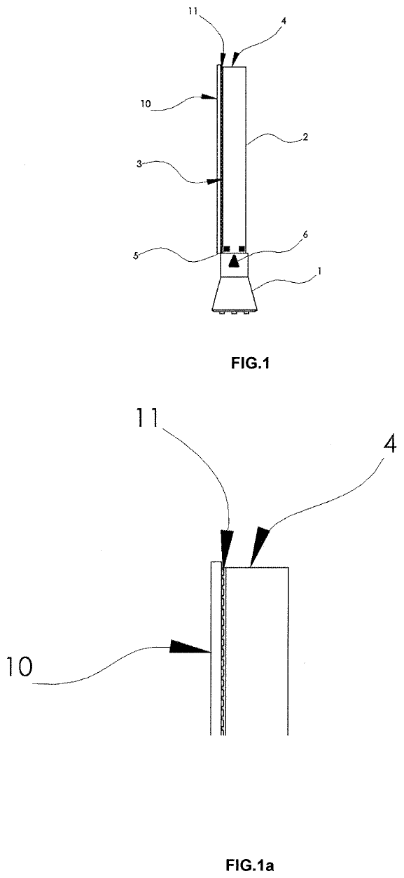

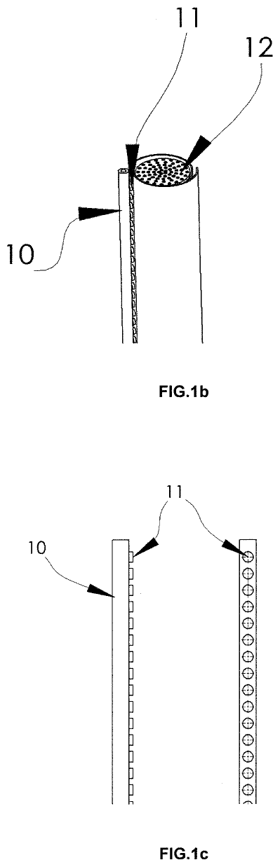

With reference to FIG. 1, which illustrates one non-restricting embodiment of a scalable, compact and low-cost CO2 removal and utilization apparatus herein disclosed. The system consists of air chamber system 2, faraday cage 12, misting column 10, nonthermal plasma generation unit consists of: electron source 5 and a pulsed directional high frequency electromagnetic beam 6. In preferred embodiment, pulsed directional high frequency electromagnetic beam 6 is a microwave emitter (e.g., magnetron), the electron source is a high-voltage electrodes pair on each side of the air chamber base.

The pulsed directional high frequency electromagnetic beam 6 can be of any electromagnetic emitting type known in the art that is able to raise the energy state for the gas molecules inside the air chamber system 2 so plasma volume can be scaled quickly inside. The pulsed directional high frequency electromagnetic beam 6 disclosed herein is capable to vibrate gas molecules in the air chamber system 2 which results in raising their energy state. In preferred embodiments, a microwave emitting unit (e.g., magnetron or solid-state microwave emitter) is employed, respectively. In some embodiments, pulsed directional high frequency electromagnetic beam can be a laser source or in other embodiment a terahertz emitting unit or an electromagnetic upconverting or down converting unit. The application in which the pulsed directional high frequency electromagnetic is employed will determine the proper source depending on required energy intensity and air chamber shape and size.

The present invention acknowledges the critical influence of the size and dimension of the plasma chamber on the microwave energy input and power consumption required for efficient operation. The dimensions of the plasma chamber play a significant role in determining the energy intensity necessary for effective plasma generation, which, in turn, is contingent upon the shape and size of the chamber. Specifically, it is recognized that:

The size and shape of the plasma chamber directly impact the distribution and concentration of the electromagnetic field generated by the electromagnetic (e.g., microwave) source. Larger chambers with varying geometries may exhibit uneven field distribution, necessitating adjustments in electromagnetic (e.g., microwave) power input to achieve uniform plasma generation throughout the chamber.

Variations in chamber size and shape influence the gas residence time within the plasma zone, affecting the extent of interaction between gas molecules and plasma species. Larger chambers typically provide longer residence times, enabling more extensive chemical reactions, but may require higher electromagnetic (e.g., microwave) energy input to sustain plasma discharge.

The electromagnetic (e.g., microwave) energy input required for plasma generation is inherently linked to the size and volume of the chamber, with larger chambers generally necessitating higher power inputs to achieve and maintain plasma states. However, optimization strategies such as adjusting gas flow rates, modifying chamber geometry, and implementing advanced plasma control techniques can help mitigate excessive power consumption while maintaining desired plasma conditions.

Consideration of the application context is paramount in selecting the appropriate electromagnetic (e.g., microwave) source, as different applications may require varying energy intensities and chamber configurations. The optimal source selection is contingent upon factors such as desired plasma characteristics, processing capacity, and energy efficiency.

It is acknowledged that plasma ignition typically requires a minimum threshold of electromagnetic (e.g., microwave) energy density (expressed in mW/cm²). Below this threshold, plasma generation may be inhibited, necessitating an additional electron source to initiate plasma formation. However, once ignited, the plasma can sustain itself even at lower energy densities than those required for initial ignition. Consequently, employing an electron source can reduce the overall energy consumption required to sustain plasma compared to relying solely on a single source (e.g., Microwave).

In the operational framework of a plasma reactor system, it is acknowledged that the initiation of plasma formation typically demands a minimum threshold of electromagnetic energy density, commonly quantified in terms of milliwatts per square centimeter (mW/cm²). Should this energy density fall below the specified threshold, the spontaneous generation of plasma may be impeded, necessitating the integration of an additional electron source to instigate the plasma ignition process. For instance, in a scenario where a plasma reactor relies solely on electromagnetic (e.g., microwave) radiation for ignition, should the energy density of the electromagnetic (e.g., microwave) radiation not meet the requisite threshold, the inclusion of an electron source becomes imperative to catalyze plasma formation. However, once plasma ignition is achieved, the self-sustaining nature of plasma enables it to persist even at energy densities lower than those essential for the initial ignition phase. By way of illustration, upon successful plasma ignition facilitated by both electromagnetic (e.g., microwave) radiation and an electron source, the plasma reaction proceeds autonomously, generating and sustaining plasma without necessitating the continual application of high-energy electromagnetic (e.g., microwave) radiation. Consequently, the dual-energy approach, incorporating both electromagnetic (e.g., microwave) radiation and an electron source, serves to optimize the energy efficiency of the plasma reactor system, ensuring sustained plasma reactions while minimizing resource consumption.

The integration of a dual source within plasma reactor systems elicits several discernible impacts on operational efficacy and functionality:

Primarily, by eliminating the requirement for elevated electromagnetic (e.g., microwave) energy levels to initiate plasma formation, this approach enables plasma ignition at substantially reduced temperatures, thereby curtailing the aggregate energy consumption necessary for both initiation and sustained plasma reactions.

Secondarily, the utilization of low-temperature plasma, facilitated by the dual-energy approach, affords the capability to synthesize chemical compounds such as methanol, a feat unattainable via conventional high-temperature single-source plasma methodologies.

Tertiary, the adoption of a dual-energy strategy engenders a heightened plasma size-to-power ratio relative to single-source plasma reactors. This augmented ratio augments the efficiency and effectiveness of plasma reactions, enabling broader and more nuanced applications across diverse industrial processes.

Ultimately, the intrinsically robust nature of dual-energy plasma reactors permits the accommodation of elevated gas flow rates compared to single-source plasma systems. This heightened capacity for gas throughput further enhances the versatility and utility of the plasma reactor, facilitating streamlined and expedited processing of gaseous substances.

The pulsed directional high frequency electromagnetic beam 6 disclosed herein has either the fundamental frequency or one of its harmonics matched to the resonant frequency of the gas or the microdroplets being targeted. In preferred embodiments, the fundamental frequency or one of its harmonics is the non-equilibrium resonance frequency for CO2. In some embodiments, the fundamental frequency or one of its harmonics is the non-equilibrium resonance frequency for Nitrogen N2 since its concentration at atmosphere about 78%. As a result, the maximum energy transfer between the electromagnetic emission and the gas molecules would happen. In preferred embodiments, CO2 gas will get excited first. In some embodiments, Nitrogen N2 will get excited first which will increase its energy to do more collisions inside the air chamber system 2 causing CO2 molecules to get excited as a result.

The present invention recognizes the importance of aligning the characteristics of the pulsed directional high frequency electromagnetic beam with the resonant frequency of the target gas molecules or microdroplets within the plasma chamber for optimized plasma generation and maintenance.

The disclosed pulsed directional high frequency electromagnetic beam, denoted as beam 6, is engineered to possess either the fundamental frequency or one of its harmonics matched to the resonant frequency of the gas or microdroplets targeted for interaction within the plasma chamber.

The resonance frequency of the target gas molecules or microdroplets is a fundamental property dependent on their molecular structure and composition. By aligning the frequency of the electromagnetic beam with the resonant frequency of the target species, enhanced energy absorption and excitation can be achieved, facilitating more efficient plasma generation and sustained interaction between the electromagnetic radiation and the targeted molecules or microdroplets.

Matching the frequency of the electromagnetic beam to the resonant frequency of the target species allows for precise control and manipulation of plasma characteristics, including plasma density, temperature, and chemical reactivity. This resonance tuning enhances the selectivity and efficiency of plasma-based processes, such as dissociation, ionization, and chemical transformation of targeted gases or microdroplets.

The resonance frequency-based tuning of the pulsed directional high frequency electromagnetic beam represents a novel and inventive aspect of the disclosed invention, offering significant advantages in terms of process efficiency, energy utilization, and scalability for various applications, including but not limited to CO2 removal/utilization, pollutant abatement, and chemical synthesis.

In preferred embodiments, the excitation of CO2 gas is prioritized within the system. This excitation process primes the CO2 molecules for subsequent interactions within the air chamber system 2. Additionally, in specific embodiments, the excitation of Nitrogen (N2) is initiated first, thereby augmenting its energy levels and facilitating heightened collision rates within the system. These collisions, in turn, induce excitation in CO2 molecules as an outcome of the energized N2 molecules interacting within the system. The cold plasma generated within the system serves to trigger and amplify these excitation mechanisms, thereby orchestrating the sequential excitation of both CO2 and N2 molecules, contributing to the overall efficacy and functionality of the system.

Moreover, in general, resonant energy transfer from N2 (or one of its chemical compounds) to CO2 is observed to be more efficient compared to energy transfer within CO2 molecules. This efficiency stems from the larger resonant collision cross-section of N2 with CO2, which is contingent upon the overlap integral between the wave-functions of the colliding molecules. The resonant collision cross-section is directly proportional to the square of the overlap integral, influenced by the relative orientation and distance of the molecules. Consequently, a larger overlap integral enhances the efficiency of energy transfer, further optimizing the resonant energy exchange between N2 and CO2 molecules within the system.

The pulsed directional high frequency electromagnetic beam 6 disclosed herein is connected to one power unit inside the base 1. In preferred embodiments, the power unit generates high power pulses to drive the pulsed directional high frequency electromagnetic beam 6. In some embodiments, the power unit generates a continuous wave (CW) or in other embodiments a modulated wave. Consequently, the pulsed directional high frequency electromagnetic beam 6 will generate an electromagnetic wave inside the air chamber system 2 based on the shape and power of the driving signal.

The power unit, situated within the base 1 of the disclosed system, functions as a centralized control apparatus responsible for the generation and modulation of electromagnetic signals directed towards both the electromagnetic (e.g., microwave) source 6 and the electron source 5. In preferred embodiments, the power unit is engineered to deliver high-power pulses meticulously optimized to energize the pulsed directional high-frequency electromagnetic beam 6. These pulses are precisely calibrated to attain maximum operational efficiency and effectiveness in propelling the electromagnetic beam.

In alternative embodiments, specific operational requirements may necessitate the output of continuous wave (CW) or modulated wave signals from the power unit, contingent upon the desired mode of operation and system functionality. In such instances, the power unit is programmed to generate electromagnetic signals of the requisite frequency and intensity to sustain the operation of the electromagnetic (e.g., microwave) source and the electron source as per the predetermined specifications of the system.

The driving mechanism of both the electromagnetic (e.g., microwave) source and the electron source is meticulously controlled by the power unit to ensure synchronized and coordinated operation within the system. The power unit governs the generation, modulation, and transmission of electromagnetic signals to these sources, optimizing their performance and functionality while upholding operational stability and dependability.

The pulsed directional high frequency electromagnetic beam 6 disclosed herein is connected to a waveguide to direct and amplify the emission before plugging it up to the air chamber system 2. In preferred embodiments, the waveguide is a microwave antenna. In some embodiments, it can be a set of lenses or in other embodiments a non-linear medium.

The disclosed system incorporates a waveguide to facilitate the efficient transmission and amplification of the pulsed directional high-frequency electromagnetic beam 6 before its introduction into the air chamber system 2. In preferred embodiments, the waveguide is configured as an electromagnetic (e.g., microwave) antenna, meticulously engineered to harness and propagate electromagnetic energy with optimal efficiency and fidelity.

Alternatively, in some embodiments, the waveguide may comprise a set of lenses, strategically arranged to focus and channel the electromagnetic beam with precision and accuracy. These lenses serve to manipulate the trajectory and distribution of the electromagnetic energy, enhancing its coherence and effectiveness within the system.

In other embodiments, the waveguide may consist of a non-linear medium, designed to modulate and amplify the electromagnetic signals in a controlled manner. By exploiting the unique properties of non-linear materials, such as their susceptibility to external stimuli and their capacity for signal amplification, the waveguide optimizes the transmission and propagation of the electromagnetic beam, ensuring its robust performance and reliability.

The selection of the waveguide configuration is contingent upon specific operational requirements and system objectives, with each embodiment tailored to maximize the efficiency and effectiveness of the electromagnetic beam coupling process. By integrating the waveguide into the system architecture, the disclosed invention achieves enhanced performance and functionality, advancing the state-of-the-art in plasma generation and manipulation technology.

For instance, in large-scale industrial settings, the utilization of aluminum waveguides represents a paradigmatic advancement. Leveraging the inherent properties of aluminum, including its high conductivity and low impedance characteristics, enables precise control and modulation of electromagnetic beam propagation. This capability facilitates optimal energy distribution and deposition, thereby enhancing the efficiency and selectivity of various processes, such as CO2 conversion reactions. Moreover, the robustness and thermal stability of aluminum waveguides ensure sustained performance under demanding operational conditions, thereby enhancing system reliability and longevity. This multifaceted approach redefines benchmarks for scalability, versatility, and performance in plasma-based technologies, underscoring the potential for widespread deployment and transformative impact across diverse industrial sectors.

The electron source 5 can be of any spark generation known in the art that is able to create freed electrons in the air chamber system 2. The electron source 5 disclosed herein is capable to start plasma in the air chamber system 2 which results in making ions and free electrons. In preferred embodiments, a high voltage electrode pair is employed, respectively. In some embodiments, the electron source can be a Tesla coil or in other embodiment a Van der Graaf generator, Marx generator or Wimshurst machine. In some other embodiments, the electron source can be a passive element (thin metals wire or sheet). The application in which the electron source is employed will determine the proper electron source depending on required energy intensity and air chamber shape and size.

The electron source 5 disclosed herein encompasses a variety of mechanisms known in the art for generating free electrons within the air chamber system 2. These mechanisms serve to initiate plasma formation within the chamber, leading to the generation of ions and free electrons essential for plasma-based processes. In preferred embodiments, the electron source may comprise a high voltage electrode pair configured to induce spark discharge within the chamber, thereby liberating electrons from the surrounding gas molecules.

Alternatively, in some embodiments, the electron source may be implemented as a Tesla coil, leveraging its capacity to produce high voltage, high frequency electrical discharges capable of ionizing the surrounding gas medium. Similarly, in other embodiments, the electron source may take the form of a Van der Graaf generator, Marx generator or a Wimshurst machine, each designed to generate and release free electrons into the chamber environment.

In some other embodiments, the electron source may consist of passive elements such as thin metal wires or sheets, strategically positioned within the chamber to facilitate electron liberation through thermal or field emission processes. The selection of the appropriate electron source is contingent upon the specific requirements of the application, including desired energy intensity, chamber geometry, and operational parameters.

The versatility and adaptability of the disclosed electron source allow for its integration into a wide range of plasma-based systems and applications, providing enhanced control and flexibility in plasma generation and manipulation processes.

In preferred embodiments, the electron source 5 disclosed herein is connected to one or more high voltage capacitors to increase the electrons flow during the discharge process. In some embodiments, Leyden jar is used for the same purpose.

In preferred embodiments, the electron source 5 disclosed herein is connected to one power unit inside the base 1. The power unit generates high voltage pulses to drive the electron source 5. In some embodiments, the power unit generates a continuous wave (CW) or in other embodiments a modulated wave. Consequently, the electron source 5 will result in ions and freed electrons with the ignition of the plasma.

In preferred embodiments, the electron source 5 disclosed herein is operatively connected to a power unit, facilitating the generation of high voltage pulses to drive the electron source 5. While the power unit may be situated within the base 1 of the system, it is not restricted to this location.

In some embodiments, the power unit is configured to generate a continuous wave (CW) signal, ensuring a steady and uninterrupted flow of electrical energy to the electron source 5. However, for enhanced control and optimization of plasma characteristics, the power unit may alternatively produce a modulated wave signal. This modulated wave signal allows for precise manipulation of plasma parameters such as density, temperature, and composition over time.

For instance, by modulating the frequency of the wave signal, it is possible to induce variations in the energy deposition rate within the plasma, thereby influencing the plasma temperature. Similarly, adjustments to the amplitude of the modulated wave can directly impact the density of charged particles within the plasma, influencing its overall density and ionization level.

Furthermore, the phase modulation of the wave signal enables temporal control over plasma ignition and extinguishment, allowing for precise synchronization with specific process requirements. By dynamically altering these modulation parameters, the plasma characteristics can be finely tuned to achieve desired outcomes in various applications, including surface treatment, material synthesis, and environmental remediation.

Consequently, the integration of modulated wave functionalities with the electron source 5 and power unit offers unparalleled versatility and control in plasma-based processes, enabling tailored and efficient plasma generation across a diverse range of industrial and scientific applications.

In other embodiments, the electron source 5 disclosed herein is connected to external power source which can feed one or more plasma reactors at the same time.

In alternative embodiments, the electron source 5 as disclosed herein is linked to an external power source distinct from the power unit situated within the base 1. This external power source is designed to supply energy to multiple plasma reactors concurrently. Unlike the localized power unit within the base 1, this external source operates at a higher level, facilitating the simultaneous activation of multiple embedded power sources. This configuration is particularly suited for array or grid connections, streamlining operational complexities by centralizing certain processes through a single external power source. By distributing power management tasks across a broader infrastructure, this approach enhances system scalability and efficiency while optimizing resource utilization within the plasma reactor network.

In preferred embodiments, the electron source unit 5 disclosed herein is connected to a flyback transformer with a feedback circuit to the main controller.

In preferred embodiments, the electron source unit 5 disclosed herein is operatively connected to a flyback transformer equipped with a feedback circuit, which interfaces with the main controller of the system. This integration serves to enhance the efficiency and reliability of the electron source unit 5 by providing real-time monitoring and regulation of key operational parameters.

The inclusion of a feedback circuit enables the continuous measurement and analysis of relevant performance metrics, such as voltage output, current levels, and waveform characteristics, associated with the flyback transformer. This data is then relayed to the main controller, which utilizes advanced algorithms and control logic to dynamically adjust the operating parameters of the flyback transformer in response to changing system conditions.

By actively monitoring and optimizing the performance of the flyback transformer in this manner, the feedback circuit ensures consistent and stable operation of the electron source unit 5 across varying load conditions and environmental factors. Additionally, it facilitates rapid detection and mitigation of potential issues or anomalies, thereby minimizing downtime and enhancing overall system reliability.

Furthermore, the integration of the flyback transformer with a feedback circuit provides enhanced flexibility and adaptability to accommodate different operational requirements and application scenarios. Through precise control and regulation of the transformer's output parameters, the system can effectively tailor the electron source unit 5 to suit specific process conditions and achieve optimal performance in diverse plasma-based applications.

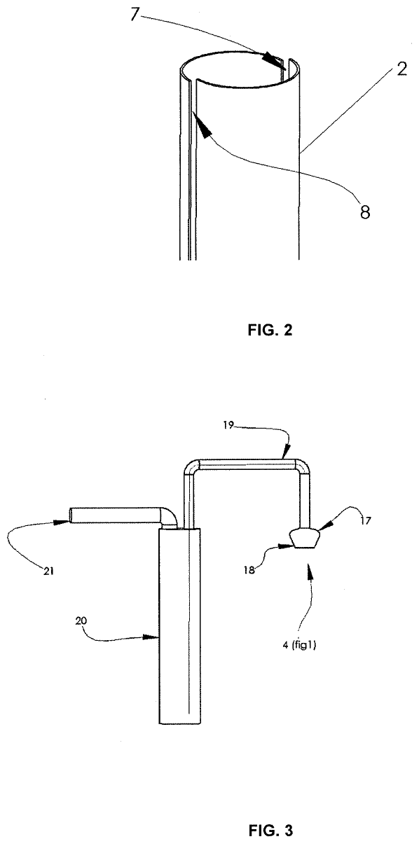

With reference made to FIG. 2, which illustrates one non-restricting embodiment of the air chamber system structure herein disclosed. The air chamber system 2 comprises of inlet openings. In preferred embodiments, multiple vertical slits are employed 7,8. In some embodiments, mostly with larger chambers, multiple large plates are aligned in parallel where their centers are fixed on a circular orbit forming multiple inlets. These parallel plates are employed to increase the performance of the system by increasing the speed of the inlet air flow.

With reference to FIG. 2, depicting a non-limiting embodiment of the air chamber system structure herein disclosed, the air chamber system 2 comprises inlet openings. In preferred embodiments, the inlet openings are characterized by multiple vertical slits 7, 8, each formed through the provision of an external casing composed of two separate parts, each featuring a precisely engineered gap. These gaps allow for the creation of the slits, which are strategically positioned to facilitate the entry of air into the chamber. The dimensions and spacing of the slits are carefully calibrated to optimize air flow dynamics while preventing unwanted disturbances.

In some embodiments, particularly in instances involving larger chambers, the inlet openings may consist of multiple large plates, characterized by dimensions typically matching the height of the chamber, arranged in parallel alignment. These plates, constructed from durable materials such as metal or composite materials, are affixed to the chamber walls in a manner that ensures stability and structural integrity. Their placement follows a circular orbit pattern, with their centers anchored to the chamber walls. This configuration results in the formation of multiple inlets distributed evenly around the circumference of the chamber.

The use of parallel plates is intended to augment system performance by enhancing the velocity of the inlet air flow. By increasing the surface area available for air entry and minimizing obstructions, the parallel plate arrangement promotes efficient air circulation within the chamber. Additionally, the design allows for flexibility in adjusting the size and spacing of the plates to accommodate variations in chamber dimensions and airflow requirements.

The dimensions and configuration of the slits and plates are determined based on specific application requirements, with considerations for optimizing air flow dynamics, minimizing pressure differentials, and maximizing energy efficiency. Computational fluid dynamics simulations and empirical testing may be employed to fine-tune the design parameters and ensure optimal performance under varying operating conditions.

The multiple vertical slits 7,8 disclosed herein are aligned in the air chamber in a way to allow creating air vortices once the air passes by them. Consequently, the inlet air will be accelerated as it enters the air chamber system 2 from opposite directions. As a result, an air column whirlwind will be generated inside the air chamber system 2.

The multiple vertical slits 7,8 disclosed herein are strategically positioned within the air chamber system 2 to induce the formation of air vortices upon passage of the inlet air. These slits are meticulously aligned along the chamber walls to optimize their aerodynamic effects. As the incoming air traverses the slits, the configuration of the chamber geometry and the placement of the slits create localized pressure differentials and disturbances, leading to the initiation of rotational motion within the airflow.

The geometry and dimensions of the slits are designed to exploit fluid dynamics principles, such as the Bernoulli effect and boundary layer interactions, to encourage the formation of vortices. By carefully adjusting the size, spacing, and orientation of the slits, the system can control the intensity and direction of the induced vortices, thereby tailoring the airflow patterns within the chamber to meet specific requirements.

As the accelerated air streams converge and interact within the chamber, they give rise to a swirling motion known as an air column whirlwind. This whirlwind phenomenon is characterized by the circulation of air around a central axis, creating a dynamic and turbulent flow pattern throughout the chamber volume. The rotational motion of the air column whirlwind serves to enhance mixing and dispersion of gases within the chamber, facilitating efficient interaction between the plasma and the surrounding air molecules.

The generation of the air column whirlwind represents a critical aspect of the air chamber system's functionality, as it plays a key role in promoting effective plasma ignition and sustaining plasma discharge. By harnessing the principles of fluid dynamics and aerodynamics, the disclosed system optimizes airflow management to achieve superior performance and operational efficiency.

The generated air column disclosed herein makes a pressure difference between the center axis of the generated air column and the atmospheric pressure outside the air chamber system 2. As a result, more air will be accelerated from the surrounding atmosphere towards the multiple vertical slits 7,8 inlet resulting in a better air throughput for the system.

The generated air column within the air chamber system 2 induces a localized pressure gradient between the central axis of the air column and the ambient atmospheric pressure external to the chamber. This pressure differential arises from the dynamic interaction between the accelerated airflow within the chamber and the surrounding atmospheric conditions. As the air column whirlwind intensifies, it establishes a region of reduced pressure along its central axis, relative to the higher atmospheric pressure outside the chamber.

The pressure difference between the interior and exterior regions of the chamber creates a driving force that promotes the inflow of additional air from the surrounding environment towards the inlet openings, particularly the multiple vertical slits 7, 8. This influx of ambient air enhances the overall airflow throughput within the system, augmenting the volume of air available for interaction with the plasma discharge.

The establishment of a pressure differential serves to optimize the efficiency and effectiveness of the air chamber system by facilitating a continuous and replenished air supply. By harnessing the principles of fluid dynamics and aerodynamics, the disclosed configuration maximizes air intake and circulation, thereby enhancing the performance and functionality of the plasma generation process.

In some embodiments, particularly in direct flue gas utilization, the inlet openings are characterized by one or more plugs in the air chamber system 2 that are directly connected to flue gas chimneys, or flue gas storage. In practical cases, a filtration unit is installed to prevent specific gases and/or particles to go inside the air chamber system 2.

In preferred embodiments, the generated air column disclosed herein forces air to stay longer inside the air chamber system 2 due to vortex shape curved movement before it goes out through the outlet 4. As a result, CO2 molecules will stay longer under the effect of nonthermal plasma which would improve the efficiency of breaking most of them into CO+O. The same result would be achieved for breaking H2O molecules and forming green fuel (e.g., methanol).

In the disclosed embodiments, the generated air column within the air chamber system 2 facilitates a prolonged residence time for the entrained air by inducing a vortex-like, curved flow pattern prior to exiting through the outlet 4. This distinctive airflow behavior, characterized by its swirling and spiraling motion, effectively extends the duration of air confinement within the chamber.

The vortex shape curved movement, imparted by the configuration of the inlet and outlet structures and the internal geometry of the chamber, promotes a controlled and deliberate trajectory for the airflow. As the air traverses through the chamber, it undergoes continuous swirling and spiraling motions, guided by the contours and boundaries of the chamber walls. This complex flow pattern results in the creation of a stable and persistent vortex within the chamber interior.

The prolonged residence time afforded by the vortex-shaped airflow allows for enhanced interaction between the air molecules and the nonthermal plasma present within the chamber. Specifically, CO2 molecules and H2O molecules remain subjected to the influence of the plasma for an extended duration, facilitating the efficient dissociation of CO2 into CO and O, as well as the conversion of H2O into green fuel such as methanol.

By optimizing the airflow dynamics within the chamber through the generation of a vortex-shaped airflow pattern, the disclosed system maximizes the efficacy of plasma-mediated chemical reactions, thereby improving the overall efficiency and performance of the plasma processing.

In some embodiments of the invention, the generated air column extends vertically above the air chamber system. The extension length depends on the length and width of the multiple vertical slits 7,8, the air chamber system 2 size and the power of the system. As a result, the effective area in breaking CO2 bonds can be larger than the air chamber system 2 which enhances the efficiency of the CO2 removal apparatus.

In preferred embodiments, a faraday cage 12, is used to shield electromagnetic waves by keeping them inside the chamber, amplifying them and reflecting them internally to form multiple standing wave nodes.

In the disclosed embodiments, a Faraday cage 12 is employed to contain and manipulate electromagnetic waves within the chamber. The Faraday cage functions as a shielding structure designed to confine electromagnetic fields generated within the chamber, thereby enhancing their intensity and spatial distribution. By encapsulating the electromagnetic waves, the Faraday cage facilitates their amplification and internal reflection, leading to the formation of multiple standing wave nodes throughout the chamber.

The Faraday cage, constructed from electrically conductive materials such as metal mesh or solid metal plates, effectively attenuates external electromagnetic interference while allowing internal electromagnetic waves to propagate freely. This shielding mechanism prevents the escape of electromagnetic energy from the chamber, promoting its concentration and accumulation within the enclosed space.

Furthermore, the geometry and configuration of the Faraday cage are optimized to facilitate the generation of standing wave patterns within the chamber. The internal surfaces of the cage are designed to reflect and redirect electromagnetic waves, promoting their constructive interference and the formation of distinct nodes and antinodes.

By harnessing the capabilities of the Faraday cage, the disclosed system maximizes the efficacy of electromagnetic wave manipulation within the chamber, enabling precise control over the spatial distribution and intensity of electromagnetic fields. This facilitates the realization of desired plasma characteristics and enhances the performance of plasma-based processes conducted within the chamber.

In preferred embodiments, the faraday cage 12 disclosed herein doesn't block air flow due to its small slits. In some embodiments of the invention, the number and size of small slits can be different along the faraday cage. As a result, an optimized air flow can be achieved.

In the disclosed embodiments, the Faraday cage 12 features a network of small slits strategically positioned across its surface to facilitate airflow while maintaining electromagnetic shielding. For example, the Faraday cage may include approximately 1000 slits, each measuring 1 millimeter in width and spaced 2 millimeters apart along the surface. These slits are arranged in a staggered pattern, with varying densities and dimensions in different regions of the cage to optimize airflow and electromagnetic containment.

The Faraday cage is engineered with precision-cut slits that allow for the passage of air molecules while effectively trapping electromagnetic waves within the chamber. By incorporating a specific arrangement of slits, such as alternating rows of different slit widths or a gradient of slit densities from top to bottom, the cage can achieve customized airflow patterns and maximize electromagnetic shielding efficiency.

This design approach ensures that the Faraday cage effectively balances the requirements of airflow management and electromagnetic containment. The carefully crafted arrangement of slits enables the cage to fulfill its dual function, providing robust electromagnetic shielding while promoting efficient ventilation throughout the chamber.

In preferred embodiments, the faraday cage 12, is constructed with one layer of metal. In some embodiments, more layers of metals or insulators can be added. In some other embodiments, at least one layer has an active metal that acts as a pre-catalyst, in-catalyst or post-catalyst where it facilitates CO2 to green fuel reactions (e.g., methanol synthesis).

In preferred embodiments, the Faraday cage 12 is fabricated using a single layer of metal, providing effective electromagnetic shielding properties. However, in alternative embodiments, additional layers of metals or insulating materials may be incorporated to enhance electromagnetic containment or insulation, respectively.

Furthermore, in certain embodiments, at least one layer of the Faraday cage comprises an active metal that serves as a pre-catalyst, in-catalyst, or post-catalyst within the plasma reaction process. This active metal component plays a pivotal role in facilitating CO2-to-green fuel (e.g., methanol) conversion reactions, either by initiating precursor transformations, catalyzing intermediate steps within the plasma environment, or promoting post-plasma chemical reactions.

The incorporation of an active metal layer within the Faraday cage structure enables synergistic interactions between electromagnetic shielding and catalytic functionality, thereby enhancing the efficiency and selectivity of CO2 conversion processes within the plasma chamber.

This integrated design approach optimizes the performance of the Faraday cage assembly, contributing to the overall effectiveness of the plasma-based CO2 conversion system.

In some embodiments of the invention, the faraday cage 12, has a magnetic layer that keeps plasma away from the walls. As a result, less plasma energy is wasted.

In certain embodiments of the invention, the Faraday cage 12 incorporates a magnetic layer strategically positioned to confine plasma within the central region of the chamber, thereby minimizing plasma energy dissipation and enhancing operational efficiency. By harnessing magnetic confinement principles, the magnetic layer exerts a force on the charged particles comprising the plasma, directing them away from the chamber walls and towards the center of the chamber. This controlled plasma confinement mechanism mitigates energy loss associated with plasma-wall interactions, allowing for more effective utilization of plasma energy for desired chemical reactions. Consequently, the incorporation of the magnetic layer within the Faraday cage structure contributes to the optimization of plasma-based processes, enhancing overall system performance and resource utilization efficiency.

The magnetic layer disclosed herein can consist of one or more discrete permanent magnets. In some other embodiments, an electric magnet (e.g., Zeeman coil) can be employed.

In some embodiments of the invention, the faraday cage 12, is split into 2 or more sections wherein each section is connected to different high voltage source. As a result, ionic wind can be formed inside the air chamber system 2 which increases the air flow of the system.

In certain embodiments of the invention, the Faraday cage structure is meticulously engineered to facilitate precise control over airflow dynamics within the air chamber system. By implementing a partitioned Faraday cage design, the chamber is effectively divided into distinct sections, with each segment connected to a dedicated high voltage source. This segmented configuration allows for independent manipulation of electric potential gradients within each section of the chamber.

Through the coordinated application of electric fields generated by the individual high voltage sources, ions within the chamber are systematically mobilized, initiating the generation of an ionic wind phenomenon. This ionic wind, characterized by directional airflow, serves to augment air circulation throughout the system, promoting efficient exchange and distribution of air within the chamber environment.

Furthermore, the partitioned Faraday cage design affords unparalleled versatility in airflow management, enabling precise adjustment of airflow patterns and velocities across different regions of the chamber. By modulating the intensity and direction of the electric fields generated by the high voltage sources, tailored airflow profiles can be achieved to accommodate diverse operational requirements and environmental conditions.

Additionally, the utilization of multiple high voltage sources facilitates fine-tuned control over airflow characteristics, allowing for dynamic adaptation to changing conditions within the chamber. This adaptive airflow management capability enhances thermal regulation and air quality control, ensuring optimal conditions for various applications and processes conducted within the chamber.

In essence, the integration of partitioned Faraday cages and multiple high voltage sources represents a sophisticated approach to airflow optimization, enabling enhanced performance and versatility in air chamber systems.

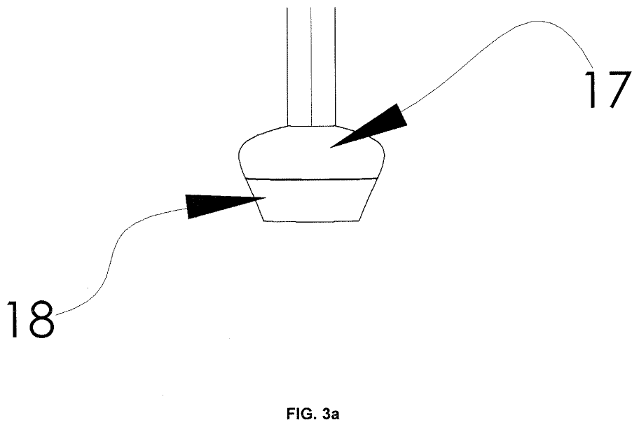

In some embodiments of the invention, with reference made to FIG. 3, which illustrates the green fuel (e.g., methanol) collection system. An ionic wind is implemented by connecting the outlet plates 17,18 to high voltage sources. As a result, an accelerated air flow would be generated from the outlet 4.

The misting column 10 disclosed herein has multiple openings wherein each opening has an ultrasonic water misting unit 11.

In certain embodiments of the invention, the misting column 10 is intricately designed to facilitate efficient and uniform dispersion of water mist within the air chamber system. The misting column comprises a series of carefully positioned openings, strategically distributed along its length to ensure comprehensive coverage of the chamber environment. Each opening is equipped with an ultrasonic water misting unit 11, configured to generate a fine mist of water droplets with precise control over particle size and distribution.

The misting column is characterized by its cylindrical shape, optimized to promote laminar flow of the water mist and minimize turbulence within the chamber. The dimensions of the misting column are tailored to suit the specific requirements of the air chamber system, with consideration given to factors such as chamber size, airflow dynamics, and desired misting coverage area. Additionally, the arrangement of openings along the length of the misting column is carefully coordinated to achieve uniform mist dispersion throughout the chamber.

Furthermore, the ultrasonic water misting units are strategically positioned within each opening of the misting column to maximize misting efficiency and coverage. Each misting unit is equipped with ultrasonic transducers capable of generating high-frequency vibrations, which in turn atomize the water into a fine mist. The size and placement of the ultrasonic transducers are optimized to ensure uniform mist generation and distribution across the entire chamber.

By integrating the misting column with ultrasonic water misting units, the air chamber system is endowed with the capability to efficiently introduce and disperse water mist, facilitating enhanced humidity control, particle suspension, and thermal regulation within the chamber environment. This meticulous design approach ensures optimal performance and reliability of the misting system, contributing to the overall effectiveness of the air chamber system for various applications and processes.

In preferred embodiments, the ultrasonic water misting unit 11 is pointed to one of the vertical slits 7,8. In some embodiments, the misting units are installed on the base of chamber. The misting column 10 disclosed herein injects microdroplets of water into the air chamber system 2. As a result, a hydrogen source is secured for green fuel (e.g., methanol) synthesis inside the air chamber system 2.

The misting column 10 disclosed herein is connected to water feed source through a pipe. In preferred embodiments, the water source is a filtered seawater that has CO2 dissolved as weak carbonic acid H2CO3. As a result, the yield of green fuel (e.g., methanol) will be increases. In some embodiments, the water source can be tape water or filtered water out of power and nuclear plants.

The nonthermal plasma generation unit disclosed herein can ignite plasma inside the air chamber system 2 either to break down CO2 into CO+O, breakdown H2O microdroplets into H+OH or to make methanol CO+4H->CH3OH.

In certain embodiments of the invention, the nonthermal plasma generation unit is configured to initiate plasma reactions within the air chamber system 2, facilitating the conversion of various molecular species for desired outcomes. Primarily, the plasma generation unit is adept at catalyzing the breakdown of CO2 molecules into CO and atomic O. This reaction, represented as CO2->CO+O, involves the dissociation of CO2 molecules under the influence of nonthermal plasma, resulting in the generation of CO and O species.

Additionally, the plasma generation unit enables the breakdown of H2O microdroplets into hydrogen (H) and hydroxyl (OH) radicals. This reaction, depicted as H2O->H+OH, involves the disintegration of water molecules into their constituent H and OH radicals through the energetic interactions facilitated by nonthermal plasma. The resulting H and OH radicals exhibit high reactivity and play pivotal roles in various chemical processes and reactions within the chamber environment.

In preferred embodiments, the plasma generation unit facilitates the synthesis of methanol (CH3OH) from CO and H species. This synthesis reaction, represented as CO+2H2->CH3OH, involves the conversion of CO and H molecules into methanol through a series of intermediate steps mediated by nonthermal plasma. The plasma environment promotes the formation of methanol molecules by facilitating the combination of CO and H species, leading to the production of methanol as a valuable chemical product within the air chamber system.

These reactions are governed by the energetic interactions between molecular species and plasma constituents, orchestrated by the plasma generation unit to achieve specific chemical transformations and desired outcomes within the chamber environment. The precise control and optimization of plasma parameters, including temperature, pressure, and energy input, play crucial roles in driving these reactions and maximizing their efficiency and yield.

In preferred embodiments, the nonthermal plasma generation unit works in a continuous mode where it sends continuous tuned plasma pulses that deliver enough energy to break CO2 and H2O and form MeOH. The pulses' power and timing properties are selected carefully to not make other unwanted chemical reactions. As a result, a digital green fuel (e.g., methanol) synthesis is achieved.

In certain embodiments of the invention, the nonthermal plasma generation unit operates in a continuous mode, emitting precisely tuned plasma pulses that deliver sufficient energy to facilitate the breakdown of CO2 and H2O molecules, subsequently leading to the formation of green fuel (e.g., methanol). The continuous mode of operation ensures a steady supply of plasma pulses, each carefully calibrated to impart the requisite energy for the desired chemical transformations while minimizing the occurrence of unwanted side reactions.

The generation of continuously tuned plasma pulses involves the utilization of advanced control mechanisms and waveform modulation techniques to tailor the properties of each pulse, including its power and timing characteristics, to suit the specific requirements of the green fuel (e.g., methanol) synthesis process. By finely adjusting these parameters, the plasma pulses can be optimized to effectively break down CO2 and H2O molecules while avoiding the initiation of undesired chemical reactions or by-products.

Furthermore, in certain embodiments, the continuous operation of the plasma generation unit enables the realization of a digital green fuel (e.g., methanol) synthesis process. Digital green fuel (e.g., methanol) synthesis refers to the precise and controlled synthesis of green fuel (e.g., methanol) molecules through the systematic modulation of plasma pulses, guided by predetermined algorithms or digital control schemes. This approach allows for the precise manipulation of plasma parameters in real-time, ensuring optimal conditions for green fuel (e.g., methanol) formation while maintaining process stability and efficiency.

Overall, the continuous generation of tuned plasma pulses, coupled with the implementation of digital control strategies, facilitates the efficient and reliable synthesis of green fuel (e.g., methanol) from CO2 and H2O molecules within the air chamber system. This digital green fuel (e.g., methanol) synthesis process represents a technologically advanced and scalable approach to green fuel (e.g., methanol) production, offering enhanced control and flexibility compared to traditional synthesis methods.

The nonthermal plasma generation unit disclosed herein is connected to measurement sensors in order to estimate the amount of CO2 being removed from atmosphere/flue gas. In preferred embodiments, one measurement sensor measures the impedance of the nonthermal plasma generated inside the air chamber system 2. Another sensor measures the green fuel (e.g., methanol) concentration over time in the water tank 20. Another sensor is employed to measure the air flow in the pipes 19. Another sensor is employed to measure the water flow in the misting column 10. Another sensor is employed to measure the forward and reflected power for the power unit connected to the pulsed directional high frequency electromagnetic beam 6 and the electron source 5. Another sensor is employed to measure water flow in the misting column 10. In some embodiments, an optical sensor (e.g., Spectrometer) is employed to measure the amount of CO2 gas inside the air chamber system 2. In other embodiments, temperature sensors are employed. The size and shape of the air chamber system 2 will determine the proper measurement sensors.

In certain embodiments of the invention, the measurement of plasma impedance involves the utilization of specialized sensors and techniques tailored to the unique properties of nonthermal plasma. One approach to measuring plasma impedance involves the use of electrical probes or electrodes positioned within the air chamber system 2, which are configured to make contact with the plasma. These probes may be designed to apply a small electrical signal to the plasma and measure the resulting current and voltage characteristics. By analyzing the relationship between the applied voltage and the resulting current, the impedance of the plasma can be determined.

Alternatively, impedance measurement techniques based on radio frequency (RF) or microwave principles may be employed. In such embodiments, RF or microwave signals are directed towards the plasma, and the reflected signals are analyzed to extract information about the impedance of the plasma. This may involve techniques such as time-domain reflectometry (TDR) or vector network analysis (VNA), which allow for precise characterization of the plasma impedance over a range of frequencies.

Furthermore, impedance measurement sensors may incorporate advanced signal processing algorithms and feedback mechanisms to dynamically adjust the operating parameters of the nonthermal plasma generation unit in response to changes in plasma impedance. This closed-loop control approach enables real-time optimization of plasma generation conditions to maximize CO2 conversion efficiency and overall system performance.

It is important to note that the measurement of plasma impedance may require careful calibration and validation procedures to ensure accuracy and reliability. Additionally, the design and placement of impedance measurement sensors within the air chamber system 2 should be optimized to minimize interference from external factors and maximize sensitivity to changes in plasma properties. Overall, the measurement of plasma impedance plays a crucial role in the effective operation and control of the nonthermal plasma-based CO2 conversion system.

The scalable, compact and low-cost CO2 removal apparatus disclosed herein has measurement sensors for CO2, green fuel (e.g., methanol) and CO that measure these gases concentration in the surrounding environment. These sensors can be of any sensor type known in the art that can measure CO2 and CO levels. (e.g., CO electrochemical sensor and NDIR infrared CO2 sensor). The main controller will turn off or on the generated nonthermal plasma based on the readings of disclosed gas measurement sensors. As a result, the CO2 and CO gas concentration can be regulated in the environment with the scalable, compact and low-cost CO2 removal apparatus is installed.

The scalable, compact and low-cost CO2 removal apparatus basic working principle can be described as follows. In any high frequency nonthermal plasma system, it is hard to generate nonthermal plasma without having an environment with either a sub-atmospheric air pressure or subsonic air pressure. This requirement makes it hard to bring an efficient CO2 removal and utilization that works at normal atmospheric pressure. This invention provides an effective way to generate nonthermal plasma at atmospheric pressure level without the need for air compressors, vacuum pumps and expensive power instruments. At first, the pulsed directional high frequency electromagnetic beam 6 emits short pulsed electromagnetic waves inside the air chamber system 2. These pulses will raise up the energy state for the gases molecules inside the air chamber system 2. The frequency of these pulses makes a perfect energy transfer to CO2 gas molecules due to resonance matching. Then the electron source 5 will generate a spark where free electrons will be launched into the air chamber system 2. Consequently, an avalanche ionization process will start inside the air chamber system 2. The air column generated inside the air chamber system 2 due to vortices implemented by the multiple vertical slits 7,8; will accelerate the avalanche/multiphoton ionization process as it increases the collisions between the free electrons and the high energy state gas molecules. As a result, most of the air column inside the air chamber system 2 will reach ionization state in a short time resulting in a nonthermal plasma which can be kept on by keeping pulsed directional high frequency electromagnetic beam 6 and electron source 5 running.

In certain embodiments, the operational mechanism of the scalable, compact, and cost-effective CO2 removal apparatus is detailed as follows. Conventional high frequency nonthermal plasma systems often require sub-atmospheric or subsonic air pressure environments to generate nonthermal plasma efficiently. However, such requirements pose challenges for achieving effective CO2 removal and utilization at normal atmospheric pressure. This invention addresses this challenge by offering a solution to generate nonthermal plasma at atmospheric pressure without the need for air compressors, vacuum pumps, or expensive power instruments.

The operational process begins with the emission of short pulsed electromagnetic waves by the pulsed directional high-frequency electromagnetic beam 6 within the air chamber system 2. These pulses elevate the energy state of gas molecules within the chamber, with the frequency of these pulses optimized for resonance matching with CO2 gas molecules. This resonance matching ensures efficient energy transfer to CO2 molecules, effectively activating them for further chemical reactions.

Simultaneously, the electron source 5 initiates a spark, releasing free electrons into the air chamber system 2. This action triggers an avalanche ionization process within the chamber, where free electrons rapidly collide with high-energy state gas molecules. The presence of vortices induced by the multiple vertical slits 7,8 accelerates this ionization process by increasing collisions between free electrons and gas molecules. Consequently, a significant portion of the air column inside the chamber rapidly transitions to an ionized state, leading to the formation of nonthermal plasma.

The degree of ionization within the air chamber system 2 is notable, with a substantial portion of the air column achieving an ionized state in a short duration. This highly ionized state facilitates the maintenance of nonthermal plasma within the chamber, with the continued operation of the pulsed directional high-frequency electromagnetic beam 6 and electron source 5 ensuring the sustained presence of nonthermal plasma.

The combined effects of resonance matching, avalanche/multiphoton ionization, and vortex-induced acceleration contribute to the rapid and efficient generation of nonthermal plasma within the air chamber system, enabling effective CO2 removal and utilization without the need for specialized equipment or sub-atmospheric conditions.

The CO2 reduction method to CO basic working principle can be described as follows. The direct dissociation energies of CO2→CO+O and CO→C+O are, respectively, 5.45 and 11.40 eV. The required energies would need a hot plasma to do such a process in atmospheric pressure. This invention employs vibrating CO2 molecules using the pulsed directional high frequency electromagnetic beam 6 and the air column generated by the vortices implemented the multiple vertical slits 7,8 inside the air chamber system; to increase the collisions. As a result, less energy is required to reduce CO2 to CO compared to direct association energy.

The CO2 reduction method to CO basic working principle can be further described as follows. The nonthermal plasma will excite some CO2 molecules inside the air chamber system 2. These excited molecules will gain energy to hit other unexcited CO2 molecules causing the first to lose their gained energy in a vibrational-translation (VT) relaxation which would depopulates the vibrational levels of CO2. The air column vortices inside the air chamber system 2 will increase the expansion of collisions to cover the whole air chamber system 2. As a result, an energy-efficient CO2 conversion into CO+O happens with an energy less than 3.7 eV due to the vibrational ladder climbing pathway.

The CO2 reduction method to CO basic working principle can be further described as follows. The broken CO and O will hit other air molecules including CO2 molecules. As a result, the vibrational depopulation process will speed up.

The CO2 reduction method to CO basic working principle can be further described as follows. Gas molecules will heat up during the described process which would result them to go up in a vortex shape due to the generated air vortices in the air chamber system 2. As a result, more collision would happen achieving a high efficiency of the system before CO+O exit from the interface (outlet) 3.

The scalable, compact and low-cost CO2 removal and utilization apparatus performance parameters can be described as follow. In some non-restricting embodiments, the air chamber system 2 has the 150 cm width and 500 cm length. Air column size inside the air chamber system can be calculated as follow:

size=πr\hat{ }2 where r is the chamber radius.

The air column size is calculated to be 12.02 m3. CO2 weight in 1 m3 of air is 0.75 g. That gives the total weight of CO2 inside the air chamber system at any given time to be 0.018 kg. The air chamber system is calculated to keep air molecules for 10 seconds inside before they leave from the outlet 3. The system efficiency is calculated to be 60% on average. To calculate how much CO2 can be removed & converted in one hour:

$$CO 2 removed per hour = 60 * \frac{60}{T} * W * \frac{E}{100}$$

where T: Air residence time inside the chamber. W: CO2 weight in chamber. E: the efficiency of CO2 reduction into CO

That gives 389.845 kg of CO2 being reduced to CO for given parameters per hour assuming there is an airflow that fills the chamber 10 times a second. As a result, more than 3410 tons of CO2 can be removed per year per unit.

The scalable, compact and low-cost CO2 removal and utilization apparatus performance parameters can be enhanced for larger air chamber systems since the generated air vortices will be bigger. As a result, a reduction of the energy required to break CO2 will occur.

The H2O breaking and methanol synthesis method follows a similar working principle to the CO2 reduction process. Here's how it works: The direct dissociation energies of H2O→H2+O and H2→H+H are, respectively, 4.83 and 4.52 eV. Traditionally, breaking water molecules into H and OH requires significant energy, often involving high temperatures or chemical processes. However, our innovation employs a pulsed directional high-frequency electromagnetic beam and air column vortices to enhance collisions between water molecules, reducing the energy needed for H2O breaking.

In accordance with certain embodiments, the method for breaking down H2O and synthesizing green fuel (e.g., methanol) operates on a principle akin to the CO2 reduction process. The fundamental steps involved are as follows: The direct dissociation energies required for the conversion of H2O into H₂+O and subsequently H2 into H+H are quantified at 4.83 and 4.52 eV, respectively. Conventionally, the dissociation of water molecules into hydrogen and hydroxyl radicals necessitates substantial energy inputs, often achieved through elevated temperatures or chemical reactions.

However, the present invention approach capitalizes on the utilization of a pulsed directional high-frequency electromagnetic beam and the induction of air column vortices to augment collisions among water molecules, thereby mitigating the energy demand for H2O dissociation. This methodology enables a more efficient and economical means of breaking down water molecules and subsequently synthesizing green fuel (e.g., methanol). By leveraging these technological advancements, we facilitate the synthesis of green fuel (e.g., methanol) from water at significantly reduced energy requirements, thereby enhancing the feasibility and sustainability of green fuel (e.g., methanol) production processes.

Specifically, the nonthermal plasma excites water molecules inside the chamber, increasing their energy and facilitating collisions with other water molecules. This process, combined with the air column vortices, accelerates the depopulation of vibrational levels in H2O, leading to efficient water molecule splitting with energy requirements lower than traditional methods like electrolysis.

Specifically, the nonthermal plasma, generated within the confines of the chamber, induces a state of excitation in water molecules by imparting additional energy to their constituent atoms. This heightened energy state renders the water molecules more reactive, increasing the likelihood of collisions with neighboring water molecules. Moreover, the presence of air column vortices within the chamber serves to enhance these collision events, effectively promoting the exchange of energy among adjacent water molecules. As a consequence of these collective phenomena, the vibrational states within the water molecules experience rapid depopulation, facilitating the efficient breaking of chemical bonds between H and OH. Notably, this process of water molecule dissociation is achieved with energy inputs that are substantially lower compared to conventional methodologies such as electrolysis, thereby underscoring the efficacy and energy efficiency of the disclosed approach.

The broken hydrogen and OH molecules resulting from the H2O breaking process will further enhance the vibrational depopulation process, speeding up the overall reaction. Additionally, the heating of gas molecules during this process creates upward air vortices, promoting more collisions and enhancing system efficiency.

In the outlined method, the disintegration of water molecules into hydrogen and hydroxyl radicals serves as a pivotal step triggering the depopulation of vibrational energy states within the system. This intricate process unfolds as follows: initially, the nonthermal plasma interacts with water molecules, inducing vibrational excitation and setting the stage for collision-induced dissociation. As this occurs, the system undergoes a cascade of vibrational energy transfer, catalyzed by the presence of the generated radicals. These radicals, in turn, act as mediators, facilitating the redistribution of vibrational energy among water molecules and promoting their subsequent dissociation. The heightened vibrational activity, augmented by the catalytic action of the radicals, accelerates the depopulation of higher-energy vibrational states, leading to a more pronounced vibrational excitation profile overall. Concurrently, the convective air currents generated by the heating of gas molecules within the chamber further amplify the vibrational dynamics, fostering an environment conducive to rapid energy exchange and molecular transformation. This intricate interplay between vibrational excitation, radical-mediated dissociation, and convective motion underscores the multifaceted nature of the proposed methodology, highlighting its efficacy in driving the conversion of water molecules into reactive species while elucidating the underlying mechanisms governing vibrational energy redistribution and depopulation.

The nature of water as microdroplets aids in plasma breakdown due to its small size, maximizing the surface area available for interaction with the nonthermal plasma. These microdroplets ensure efficient energy transfer, enabling the plasma to break down water molecules effectively.

Unlike water vapor, which consists of individual molecules dispersed in the air, water microdroplets offer a concentrated form of water that is more conducive to plasma interactions. By utilizing water microdroplets, rather than water vapor, the system optimizes the efficiency of H2O breaking and green fuel (e.g., methanol) synthesis, leading to higher yields and lower energy requirements.

The performance parameters of the scalable, compact, and low-cost apparatus for H2O breaking and green fuel (e.g., methanol) synthesis are calculated similarly to those for CO2 reduction. With optimized chamber sizes and energy-efficient processes, the system can achieve substantial CO2 removal/utilization and green fuel (e.g., methanol) synthesis rates, offering a promising solution to address climate change and fuel production challenges.

As the system's capabilities are further scaled up for larger chamber systems, the generated air vortices become more significant, resulting in even lower energy requirements for H2O breaking and green fuel (e.g., methanol) synthesis. This scalability allows for the removal and utilization of significant CO2 quantities and green fuel (e.g., methanol) production on a larger scale, contributing to a cleaner and more sustainable future.

To enhance green fuel (e.g., methanol) production, the misting process can utilize a CO2-rich water source, such as seawater. Oceans serve as significant CO2 sinks globally, but the increasing concentration of dissolved CO2 in seawater poses a threat to marine life. The present invention could potentially be employed to extract CO2 from oceans. CO2 typically dissolves in seawater as carbonic acid (H₂CO₃), a weak acid that can be broken down into CO2 and H2O by the plasma, serving as the primary inputs of the core system.

With reference to FIG. 3, which illustrates one non-restricting embodiment of green fuel (e.g., methanol) collection system; After the green fuel (e.g., methanol) gas is produced within the chamber, it is directed through a pipe 19 that leads to the bottom of a water tank 20. As the green fuel (e.g., methanol)-laden gas rises through the water column, the green fuel (e.g., methanol) quickly dissolves into the water due to its high solubility, while other atmospheric/flue gas gases bubble and escape from the surface of the water through the tank outlet 21 to atmospheric/flue gas air or to other tanks doing a similar or a different process. The dissolved green fuel (e.g., methanol) forms a solution within the water tank 20.

To capture the dissolved green fuel (e.g., methanol), multiple water tanks can be used in series. As the green fuel (e.g., methanol)-laden gas passes through each tank, more green fuel (e.g., methanol) is absorbed into the water, increasing the concentration of green fuel (e.g., methanol) solution.

Once the water tanks have captured the green fuel (e.g., methanol), the solution can undergo distillation to extract pure green fuel (e.g., methanol) from the water. Distillation involves heating the green fuel (e.g., methanol)-water solution to separate the green fuel (e.g., methanol) vapor, which is then condensed back into liquid form. This process allows for the extraction of pure green fuel (e.g., methanol), which can be collected and further processed for various applications.

The scalable, compact and low-cost CO2 removal apparatus disclosed herein utilizes the measurement systems for plasma impedance, power and other environment parameter to calculate how much CO2 is being removed or utilized. In preferred embodiments, a local digital signal processor (DSP) calculates the removed CO2 quantity, generates a blockchain entity by hashing this value with device ID and other parameters, stores it in a local memory before connecting to cloud blockchain service for authenticated tokenization.

The fully digital and secure tokenization of the removed CO2 renders it suitable to replace existing Measurement, Reporting, and Verification (MRV) of Carbon Credits that being implement widely these days on businesses and governments level to regulate the CO2 emissions globally. Existing systems rely on offline verification and 3rd party reports to quantify and validate the amount of removed CO2. This invention leverages the power of blockchain and direct removal CO2 measurement methods disclosed above to empower the MRV system and scale it up globally.

The scalable, compact, and low-cost CO2 removal and utilization apparatus renders it suitable for large-scale deployment to remove CO2 from the air due to its low cost, low energy requirement, and high efficiency. This invention doesn't require pre/post-treatment, chemical processes, high temperature, or consumables. The byproduct, e.g., methanol, serves as a clean fuel for various applications, contributing to a sustainable energy ecosystem.

In view of the above, it will be appreciated that the present invention also relates to a method, an apparatus and/or a nonthermal plasma generation unit with any, some or all of the following features. The apparatus and/or the nonthermal plasma generation unit may be implemented such that some of the respective features disclosed herein are combined with one another, unless indicated otherwise or technically inappropriate.

An apparatus to remove or utilize CO2 directly from air/flue gas and generate green fuel (e.g., methanol) may comprise one or more selected from the group consisting of: an air chamber system; a nonthermal plasma generation unit, a faraday cage, a misting column, a green fuel (e.g., methanol) collection system, one or more measurement sensors, a controller, a data storage unit, a communication module and a power supply unit.

The air chamber system may be configured to break CO2 bonds and forming green fuel (e.g., methanol) comprising of a group of inlets and outlets. The air chamber system may also be referred to as plasma chamber, unless indicated otherwise.

A nonthermal plasma generation unit may comprise an emitter and/or a charge carrier source. The emitter may be a high frequency electromagnetic emitting unit. The charge carrier source may be an electron source, and particularly an electron ignition source.

The nonthermal plasma generation unit may raise, or may be configured to raise, the energy state for the gas inside the air chamber system.

The nonthermal plasma generation unit may ionize, or may be configured to ionize, the gas inside the air chamber system.

The nonthermal plasma generation unit may generate, or may be configured to generate, a non-equilibrium plasma at atmospheric pressure.

the nonthermal plasma generation unit may generate, or may be configured to generate, an equilibrium plasma at atmospheric pressure.

The nonthermal plasma generation unit may be configured to break CO2 bonds using energy relaxation in V-T transition at different vibration levels below CO2 molecular direct dissociation.

The high frequency electromagnetic emitting unit may comprise a laser.

A fundamental frequency of the high frequency electromagnetic emitting unit may match one of the resonance frequencies of CO2.

One or more frequency harmonics of the emitter may match one of the resonance frequencies of CO2.

A frequency of the high frequency electromagnetic emitting unit may match one of the resonance frequencies of N2 or one of its chemical compounds.

One or more frequency harmonics of the emitter may match one of the resonance frequencies of N2 or one of its chemical compounds.

The emitter may be configured to induce vibrational-vibrational (VV) energy transfer between CO2 molecules. This may facilitate a dissociation of CO2 to CO and O.

The emitter may be configured to promote vibrational-translational (VT) relaxation of excited CO2 molecules. This may lead to a fragmentation of CO2 to CO and O.

The emitter may be configured to accelerate collisions between CO2 molecules. This may enable a direct conversion of CO2 to CO and O.

The emitter may be configured to facilitate direct energy transfer from excited CO2 molecules to neighboring molecules. This may result in dissociation of CO2 to CO and O.

The emitter may be configured to induce vibrational-vibrational (VV) energy transfer between H2O molecules. This may facilitate a dissociation of H2O to H2 and O.

The emitter may be configured to promote vibrational-translational (VT) relaxation of excited H2O molecules. This may lead to a fragmentation of H2O to H2 and O.

The emitter may be configured to accelerate collisions between H2O molecules. This may enable a direct conversion of H2O to H2 and O.

The emitter may be configured to facilitate direct energy transfer from excited H2O molecules to neighboring molecules. This may result in dissociation of H2O to H2 and O.

The emitter may be configured to induce vibrational-vibrational (VV) energy transfer between CO and H2 molecules. This may facilitate their combination into green fuel (e.g., methanol).

The emitter may be configured to promote vibrational-translational (VT) relaxation of excited CO and H2 molecules. This may lead to a combination of CO and H2 molecules to green fuel (e.g., methanol).

The emitter may be configured to induce vibrational-vibrational (VV) energy transfer between H+OH molecules. This may facilitate a conversion of H+OH molecules to H2 and O.

The emitter may be configured to promote vibrational-translational (VT) relaxation of excited H and OH molecules. This may lead to a conversion of H+OH molecules to H2 and O.

The emitter may be configured to accelerate collisions between H+OH molecules. This may enable a direct conversion of H+OH molecules to H2 and O.

The emitter may be configured to facilitate direct energy transfer from excited H+OH molecules to neighboring molecules. This may result in a dissociation of H+OH molecules to H2 and O.

The emitter may operate in a pulsed mode.

The emitter may operate in a continuous mode (CW).

The emitter may be or comprise one or more selected from the group consisting of: a semiconductor-based laser, a gas laser, a liquid laser, a fiber laser, a solid-state laser, an x-ray emitter, an infrared emitter, an acousto-optic modulated laser, a terahertz emitter, a magnetron, a microwave solid-state generation unit, a halogen lamp, a UV diode, a terahertz emitter, and a Xenon arc lamp.

The emitter may be configured to generate electromagnetic wave in a very high frequency range. In addition, the emitter may comprise a frequency down converting unit.

The emitter may be configured to generate electromagnetic wave in a low high frequency range. In addition, the emitter may comprise a frequency up converting unit.

The charge carrier source may be or comprise at least one selected from the group consisting of: a tungsten filament, a thin metal sheet, a thin metal wire, a high voltage electrode pair, an ion source, a tesla coil, a Van der Graaf generator, a Wimshurst machine, Marx generator, a high voltage generator and a piezoelectric element.

Additionally or alternatively, the charge carrier source may be configured to emit electrons, which may contribute to ignition (i.e., initiation) of (nonthermal) plasma, particularly at atmospheric pressure.

For example, the charge carrier source may comprise a field emission cathode to emit electrons.

The charge carrier source may utilize a photoemission process to emit electrons for initiation of plasma.

The charge carrier source may include a thermionic emitter configured to emit electrons for initiation of plasma.

The charge carrier source may comprise a cold cathode electron emitter to emit electrons for initiation of plasma.

The charge carrier source may utilize a radioactive material configured to emit electrons and/or radiation for initiation of plasma.

The charge carrier source may comprise a laser beam directed onto a photocathode to emit electrons for initiation of plasma.

The charge carrier source may be connected to one or more inductors.

The charge carrier source may be connected to one or more capacitors.

The charge carrier source may be or comprise a mechanical spark ignition unit.

The communication module may be wireless or wired.

The data storage unit may be volatile or non-volatile.

The data storage unit may be or comprise one or more selected from the group consisting of: a USB flash drive a SD card and a hard disk drive,

The wireless network protocol may be or comprise one or more selected from the group consisting of: Wi-Fi, Bluetooth, Zigbee, Z-wave, 6LoWPAN, RFID, Cellular, NB-IoT, 5G, 6G, NFC, LoRaWAN, LTE-M and LPD433.

The wireless network may be or comprise a mobile data module and/or an optical module.

The wired network protocol may be or comprise one or more selected from the group consisting of: Ethernet, Serial, USB, Parallel, and a channel pair or a plurality of physical channel pairs.

The power supply unit may be powered by a sustainable energy source.

The power supply unit may be or include a battery.

The power supply unit may include one or more capacitors.

The power supply unit may be connected with, or comprise, solar panels.

At least one of the measurement sensors may be configured to measure an impedance of the plasma inside the air chamber system.

The measured impedance value may be used to estimate the amount of CO2 being removed and/or utilized.

At least one of the measurement sensors may be configured to measure the power usage of the apparatus.

At least one of the measurement sensors may be configured to measure the temperature of the apparatus.

At least one of the measurement sensors may be configured to measure air flow of the air chamber system.

At least one of the measurement sensors may be configured to measure the electron temperature inside the chamber.

At least one of the measurement sensors may be configured to measure water flow of the misting column.

At least one of the measurement sensors may be configured to measure a byproduct (e.g., methanol) concentration.

At least one of the measurement sensors may be configured to measure the humidity inside the air chamber system.

The measurement sensors may include one or more selected from the group consisting of: a camera, a color sensor, an infrared-based CO2 detector, a CO sensor, a green fuel (e.g., methanol) sensor and a Hydrogen sensor.

At least one of the measurement sensors may be configured to measure a power usage of the apparatus.

At least one of the measurement sensors may be configured to measure a forward power of the nonthermal plasma generation unit.

At least one of the measurement sensors may be configured to measure a reflected power of the nonthermal plasma generation unit.

The apparatus may comprise an air chamber system having a cylindrical general shape. The air chamber system may have inlets formed as openings or vertical slits along an axial direction of the cylindrical general shape. The air chamber system may have an outlet at one of the end surfaces of the cylindrical general shape.

A method to generate an air vortex inside the air chamber system may be provided. The air vortex may be generated as air enters the air chamber system through the inlets provided as vertical slits.

In the method, the air vortex may move along the axial direction of the cylindrical general shape of the air chamber system, in particular in an upward direction, as the air inside the air chamber system ascends while being heated up by the plasma in the air chamber system.

The air chamber system may comprise a faraday cage on an inner side in a radial direction of the cylindrical general shape.

Alternatively, or additionally, the air chamber system may comprise a faraday cage on an outer side in the radial direction of the cylindrical general shape.

The air chamber system may comprise a catalyst placed at the outlet.

The air chamber system may comprise an air filter at the outlet.

The air chamber system may comprise a carbon filter at the outlet.

The air chamber system may comprise an air filter at one, some or all of the inlets.

The apparatus may comprise a misting system to provide liquid mist, particularly water mist, to the inside of the air chamber system. The misting system may comprise a misting column that is (fluidly) connected with a water misting source.

The misting system may be configured to use provide mist of seawater to the inside of the air chamber system. Particularly, the misting system may be configured to increase the concentration of dissolved CO2. This may increase they yield of green fuel (e.g., methanol) synthesized from atmospheric/flue gas CO2.

The misting system may be configured to filter and/or purify a liquid prior to providing the liquid to the inside of the air chamber system. This may contribute to remove impurities from the liquid being used for green fuel (e.g., methanol) synthesis. Herein, the liquid may be or contain water, particularly seawater.

The misting system may be configured to pre-heat the liquid to a predetermined temperature. This may increase the efficiency of the green fuel (e.g., methanol) synthesis within the air chamber system.

The apparatus may be configured to apply ultrasonic agitation or cavitation to the liquid provided to the inside of the air chamber system. This may contribute to formation of microdroplets for more effective interaction with the nonthermal plasma during green fuel (e.g., methanol) synthesis.

The liquid provided to the inside of the air chamber system may be supplemented with additives or catalysts. This may facilitate the conversion of atmospheric/flue gas CO2 into green fuel (e.g., methanol) within the air chamber system.

The liquid may be aerated or oxygenated to improve the dissolution of CO2 and enhance the efficiency of green fuel (e.g., methanol) synthesis during plasma-assisted CO2 conversion.

The liquid may be pressurized before being provided to the inside of the air chamber system This may increase the kinetic energy of the microdroplets and promoting more efficient green fuel (e.g., methanol) synthesis.

The liquid may be pulsed or intermittently sprayed into the air chamber system to control the rate of green fuel (e.g., methanol) synthesis. This may be utilized to optimize process parameters.

The liquid may be enriched with isotopes or specific chemical species to tailor the properties of the resulting green fuel (e.g., methanol) product. This may be utilized for specific applications or markets.

The misting system may comprise an ultrasonic transducer configured to generate ultrasonic vibrations in the water to produce microdroplets for interaction with the nonthermal plasma during green fuel (e.g., methanol) synthesis.

The misting system may comprise a piezoelectric device configured to induce mechanical vibrations in the water to generate microdroplets for introduction into the plasma chamber for green fuel (e.g., methanol) synthesis.

The misting system may comprise a pneumatic atomizer configured to disperse pressurized air into the water to produce a mist of microdroplets for delivery into the plasma chamber during green fuel (e.g., methanol) synthesis.

The misting system may comprise a centrifugal atomizer configured to spin the water at high speeds to generate microdroplets for injection into the plasma chamber for green fuel (e.g., methanol) synthesis.

The misting system may comprise a vibrating mesh nebulizer configured to oscillate a fine mesh membrane to produce a fine mist of microdroplets from the water for introduction into the plasma chamber during green fuel (e.g., methanol) synthesis.

The misting system may comprise a capillary array configured to draw water through a series of fine capillaries to generate microdroplets for dispersion into the plasma chamber during green fuel (e.g., methanol) synthesis.

The misting system may comprise a sonic nozzle configured to generate acoustic waves in the water to produce microdroplets for delivery into the plasma chamber for green fuel (e.g., methanol) synthesis.

The misting system comprise a bubble column configured to produce bubbles in the water, which burst to generate microdroplets for introduction into the plasma chamber during green fuel (e.g., methanol) synthesis.

The misting system comprise an electrospray nozzle configured to apply an electric field to the water to induce the formation of charged microdroplets for injection into the plasma chamber during green fuel (e.g., methanol) synthesis.

The misting system may comprise a microfluidic device configured to manipulate the flow of water through microchannels to produce microdroplets for dispersion into the plasma chamber during green fuel (e.g., methanol) synthesis.

The apparatus may comprise a Faraday cage configured to contain electromagnetic plasma.

The Faraday cage may be designed to sustain standing waves within the plasma.

The Faraday cage may include slits or perforations to allow an ingress of air while preventing the egress of electromagnetic radiation.

The Faraday cage may be made of a conductive material. The Faraday cage may be configured to shield external electromagnetic radiations.

The Faraday cage may be positioned around the nonthermal plasma generation unit.

The Faraday cage may be configured to enclose the plasma.

The Faraday cage may comprise a mesh or grid structure to provide electromagnetic containment while allowing airflow.

The Faraday cage may be configured to promote a resonance of the plasma. This may contribute to an increase of energy efficiency.

The Faraday cage may be adapted to minimize electromagnetic leakage while facilitating the exchange of gases with the surrounding environment.

The Faraday cage may be equipped with adjustable slits or apertures to control the airflow and electromagnetic confinement within the system.

The Faraday cage may be made of a conductive material selected from the group consisting of copper, steel, stainless steel, aluminum, silver, and gold.

The Faraday cage may comprise a composite material incorporating conductive elements and catalytic components.

The Faraday cage may comprise a coating or a layer of catalytic material on its inner surface to promote chemical reactions within the plasma.

The Faraday cage may be coated with a thin film of platinum, palladium, or other noble metals to act as a catalyst for CO2 conversion reactions.

The Faraday cage may comprise nanoparticles of transition metals or metal oxides dispersed within its structure to enhance catalytic activity.

The Faraday cage may be composed of a porous material capable of adsorbing and activating reactant molecules to facilitate plasma-assisted reactions.

The Faraday cage may be fabricated from a ceramic material such as alumina or zirconia, doped with metal ions to catalyze CO2 conversion reactions.

The Faraday cage may comprise a composite material incorporating zeolites, activated carbon, or other porous substrates to adsorb CO2 and facilitate its conversion within the plasma.

The Faraday cage may comprise a reactive coating or surface treatment designed to selectively promote certain chemical reactions while inhibiting others.

The Faraday cage may be engineered to provide a tailored microenvironment conducive to specific catalytic processes, including pre-plasma activation, in-plasma conversion, and post-plasma treatment of reaction products.

The method may comprise directing the synthesized green fuel (e.g., methanol) gas to a bottom of a water tank. For example, the directing may be performed through a pipe.

The method may comprise dissolving the green fuel (e.g., methanol) gas in water while enabling other atmospheric/flue gases to escape, for example through bubbling.

The method may comprise employing a distillation technique connected to the water tank to extract (pure) green fuel (e.g., methanol) from water. This may increase the efficiency of green fuel (e.g., methanol) recovery.

The apparatus may comprise a water tank positioned below the air chamber system. The water tank may be configured to collect synthesized green fuel (e.g., methanol) gas.

The method may comprise arranging multiple water tanks in series to increase green fuel (e.g., methanol) capture efficiency.

The apparatus may comprise means for directing the green fuel (e.g., methanol) gas into the water tank(s). This may facilitate rapid dissolution.

The apparatus may comprise a distillation apparatus connected to the water tank(s) to extract pure green fuel (e.g., methanol) from the water.

The apparatus may comprise a collection chamber positioned downstream of the plasma chamber to receive a green fuel (e.g., methanol)-containing gas stream.

The apparatus may comprise a water tank located within the collection chamber to capture the green fuel (e.g., methanol) gas, solid or liquid.

The apparatus may comprise a distillation unit coupled to the water tank to separate and recover pure green fuel (e.g., methanol) from the water.

The apparatus may comprise a control mechanism configured to regulate a flow of green fuel (e.g., methanol) gas. This may be utilized to optimize green fuel (e.g., methanol) recovery efficiency.

The apparatus may comprise a filtration unit connected to the water tank(s) to remove impurities and particulates from the water.

The apparatus may comprise an adsorption unit connected to the water tank(s) to selectively adsorb green fuel (e.g., methanol) molecules from the water.

The apparatus may comprise a membrane separation unit connected to the water tank(s) to separate green fuel (e.g., methanol) molecules from water molecules based on their size and properties.

The apparatus may comprise a condensation unit connected to the water tank(s) to condense green fuel (e.g., methanol) vapors into liquid methanol for collection and recovery.

The apparatus may comprise a molecular sieve unit connected to the water tank(s) to selectively adsorb methanol molecules based on their size and properties.

The apparatus may comprise an evaporation unit connected to the water tank(s) to evaporate water and leave behind concentrated methanol solution for subsequent purification.

The apparatus may comprise a solvent extraction unit connected to the water tank(s) to extract green fuel (e.g., methanol) from the water using a suitable solvent.

The apparatus may comprise a crystallization unit connected to the water tank(s) to induce crystallization of green fuel (e.g., methanol) for separation from water.

The apparatus may comprise an ion exchange unit connected to the water tank(s) to exchange ions and separate green fuel (e.g., methanol) from water based on their ionic properties.

The apparatus may comprise a centrifugation unit connected to the water tank(s) to separate green fuel (e.g., methanol) from water based on their density difference through centrifugal force.

The apparatus may comprise a catalyst at the outlet of the air chamber system to convert the green fuel (e.g., methanol) that is produced in the first step into a higher-level hydrocarbon green fuel like green diesel and sustainable aviation fuel (SAF). This catalyst may have a shape that consists of porous structure. This catalyst may be passive or it may be activated by electrical charge, heat, thermal and/or nonthermal plasma.

The apparatus may comprise an ionic wind generator positioned within the air chamber system to induce air movement towards the water tank(s) using ionic propulsion.

The apparatus may comprise an electroosmotic flow system integrated into the air chamber system to drive air flow towards the water tank(s) through the application of an electric field.

The apparatus may comprise one or more fans positioned within the air chamber system to generate airflow towards the water tank(s).

The apparatus may comprise a venturi system integrated into the air chamber system to create suction and accelerate air flow towards the water tank(s).

The apparatus may comprise an electromagnetic induction system positioned within the air chamber system to induce air movement towards the water tank(s) through the generation of electromagnetic fields.

The apparatus may comprise a piezoelectric actuator system integrated into the air chamber system to generate mechanical vibrations and promote air flow towards the water tank(s).

The apparatus may comprise a thermal convection system configured to harness temperature differentials within the air chamber system to drive air flow towards the water tank(s).

The apparatus may comprise an acoustic resonance system positioned within the air chamber system to generate sound waves and induce air movement towards the water tank(s).

The apparatus may comprise a heating element integrated into the water tank(s) to facilitate green fuel (e.g., methanol) distillation through the application of heat.

The apparatus may comprise a microwave distillation system configured to heat the water tank(s) and facilitate green fuel (e.g., methanol) distillation using microwave energy.

The apparatus may comprise a distillation system configured to recycle any heat generated by the air chamber system or the nonthermal plasma generation unit.

The apparatus may comprise a filtration system positioned within the water tank(s) to remove impurities and separate methanol from the water through filtration.

The apparatus may comprise a centrifugal distillation apparatus coupled to the water tank(s) to separate green fuel (e.g., methanol) from water through centrifugal force.

The apparatus may comprise a vacuum distillation system configured to lower the boiling point of green fuel (e.g., methanol) and facilitate its separation from water under reduced pressure.

The apparatus may comprise a membrane distillation unit integrated into the water tank(s) to selectively permeate green fuel (e.g., methanol) vapor through a semipermeable membrane, enabling its separation from water.

The apparatus may comprise a cryogenic distillation system designed to cool the water tank(s) to temperatures below the freezing point of water, allowing for the separation and collection of green fuel (e.g., methanol) as a liquid.

The apparatus may comprise an adsorption distillation unit configured to adsorb green fuel (e.g., methanol) vapor onto a solid adsorbent material, followed by desorption to recover pure green fuel (e.g., methanol).

A method for dissociating CO2 using non-equilibrium plasma, wherein vibrational modes of CO2 are excited, followed by vibrational-vibrational (VV) energy exchange processes leading to the spreading of vibrational quanta and subsequent dissociation into CO and O.

A method for CO2 dissociation utilizing non-equilibrium plasma, comprising inducing electron impact vibrational excitation/de-excitation from ground level u0 to upper vibrational levels ui, electronic excitation from ground level u0 to e2, dissociation from ground level u0 to CO and O, and ionization from ground level u0 to CO2+.

An apparatus for CO2 dissociation under non-equilibrium conditions, comprising a plasma generation unit configured to induce vibrational excitation of CO2 molecules and subsequent dissociation into CO and O, wherein the apparatus further includes a faraday cage to contain electromagnetic plasma and enable standing waves.

An apparatus for CO2 dissociation wherein the faraday cage is made of a material that acts as a pre, in, and post-plasma catalyst to facilitate the dissociation reactions.

A method for green fuel (e.g., methanol) synthesis from CO2 using non-equilibrium plasma, comprising promoting electron impact dissociation of CO2 molecules and subsequent hydrogenation steps to convert CO and O into green fuel (e.g., methanol).

A method for green fuel (e.g., methanol) synthesis utilizing non-equilibrium plasma, wherein active hydrogen species generated by the plasma facilitate the reduction of CO2 to green fuel (e.g., methanol) through successive hydrogenation steps.

An apparatus for green fuel (e.g., methanol) synthesis from CO2 under non-equilibrium conditions, comprising a plasma generation unit configured to generate active hydrogen species and promote electron impact dissociation of CO2 molecules, wherein the apparatus further includes a collection chamber to capture synthesized green fuel (e.g., methanol) gas.

An apparatus for green fuel (e.g., methanol) synthesis may comprise a water tank positioned within the collection chamber to dissolve green fuel (e.g., methanol) gas quickly in water, allowing for efficient green fuel (e.g., methanol) recovery.

A method for enhancing green fuel (e.g., methanol) synthesis efficiency using non-equilibrium plasma, comprising modulating discharge parameters such as voltage, frequency, and waveform to optimize the yield of green fuel (e.g., methanol) production from CO2.

An apparatus for green fuel (e.g., methanol) synthesis under non-equilibrium conditions, comprising a misting unit configured to inject microdroplets of water into the plasma chamber to enhance green fuel (e.g., methanol) synthesis efficiency by promoting interactions between water vapor and CO2 molecules.

An apparatus for green fuel (e.g., methanol) synthesis similar to above with an input of carbon monoxide (CO) as a concentrated source or diluted source (flue gas or air).

An apparatus for green fuel (e.g., methanol) synthesis similar to above with an input of CO mixed with CO2 as a concentrated source or diluted source (flue gas or air).

1. An apparatus comprising:
a chamber;
an emitter configured to emit electromagnetic radiation into the chamber; and
an igniter configured to provide energy at an ignition point within the chamber to initiate a gas discharge,
wherein the emitter and the igniter are operable in conjunction to generate nonthermal plasma within the chamber at atmospheric pressure.

2. The apparatus of claim 1,
wherein the chamber has a columnar structure extending along a longitudinal axis and comprises a sidewall extending around the longitudinal axis.

3. The apparatus of claim 2,
wherein the chamber has a substantially cylindrical geometry around the longitudinal axis and extends by a height of along the longitudinal axis.

4. The apparatus of claim 2,
wherein the sidewall of the chamber is made of conductive material so as to shield an interior of the chamber from external electric fields.

5. The apparatus of claim 2,
wherein the sidewall of the chamber is gas-permeable by being at least one of: at least partially perforated, partially discontinuous, or partially open.

6. The apparatus of claim 1,
wherein the igniter comprises an electron source to generate free electrons at the ignition point by applying an electrical field.

7. The apparatus of claim 1,
wherein the chamber is configured to support a standing wave of the electromagnetic radiation emitted by the emitter.

8. The apparatus of claim 1,
wherein the emitter and the igniter are configured such that the ignition point of the igniter approximately coincides with a field maximum of the electromagnetic radiation emitted by the emitter.

9. The apparatus of claim 1,
wherein the emitter is configured to emit the electromagnetic radiation in a pulsed manner, in a continuous manner, or in an alternating combination of both.

10. The apparatus of claim 1,
wherein the emitter is configured to emit the electromagnetic radiation in a wavelength range from 100 nanometers to 1 meter.

11. The apparatus of claim 1, further comprising:
a liquid injector configured to inject a liquid into the chamber.

12. The apparatus of claim 1,
wherein the chamber comprises an outlet configured to discharge gas stream from the chamber,
wherein the apparatus further comprises a filter disposed at the outlet of the chamber and configured to capture at least one of: carbon oxides, nitrogen oxides, and sulfur oxides.

13. The apparatus of claim 1, further comprising:
a water tank in fluid communication with the chamber and configured to dissolve methanol from gas stream discharged from the chamber.

14. A system, comprising:
an air chamber system comprising a sidewall made of a conductive material and surrounding a longitudinal axis, the air chamber system being configured to be permeable to gas;
a nonthermal plasma generation unit configured to generate nonthermal plasma within a volume surrounded by the sidewall of the air chamber system by means of an electromagnetic radiation in a wavelength range of 100 nanometers to 1 meter and an igniter configured to induce gas discharge; and
a fluid injector configured to introduce a fluid into the volume surrounded by the sidewall of the air chamber system.

15. A method of plasma-assisted processing of air, comprising:
generating nonthermal plasma in a chamber at atmospheric pressure, the chamber being configured to permit airflow through the chamber;
injecting water into the chamber while maintaining the nonthermal plasma in the chamber and permitting airflow through the chamber.
